SEQUENCE LISTING (46 sequences)

SEQ ID NO: 1 - protein, 108 aa, Homo sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, Homo sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 5 aa, Homo sapiens
RYWMS

SEQ ID NO: 4 - protein, 17 aa, Homo sapiens
NIKQDGSEKYYVDSVKG

SEQ ID NO: 5 - protein, 12 aa, Homo sapiens
EGGWFGELAFDY

SEQ ID NO: 6 - protein, 12 aa, Homo sapiens
RASQRVSSSYLA

SEQ ID NO: 7 - protein, 7 aa, Homo sapiens
DASSRAT

SEQ ID NO: 8 - protein, 9 aa, Homo sapiens
QQYGSLPWT

SEQ ID NO: 9 - protein, 107 aa, Mus musculus
DIVMTQSHKLMSTSVGDRVSITCKASQDVGTAVAWYQQKPGQSPKLLIYWASTRHTGVPDRFTGSGSGTDFTLTISNVQSEDLADYFCQQDSSYPLTFGAGTKVELK

SEQ ID NO: 10 - protein, 118 aa, Mus musculus
EVKLQESGPSLVKPSQTLSLTCSVTGYSITSDYWNWIRKFPGNKLEYVGYISYTGSTYYNPSLKSRISITRDTSKNQYYLQLNSVTSEDTATYYCARYGGWLSPFDYWGQGTTLTVSS

SEQ ID NO: 11 - protein, 107 aa, Mus musculus
DIVTTQSHKLMSTSVGDRVSITCKASQDVGTAVAWYQQKPGQSPKLLIYWASTRHTGVPDRFTGSGSGTDFTLTISNVQSEDLADYFCQQDSSYPLTFGAGTKVELK

SEQ ID NO: 12 - protein, 120 aa, Mus musculus
EVQLQESGPGLVAPSQSLSITCTVSGFSLTTYSINWIRQPPGKGLEWLGVMWAGGGTNSNSVLKSRLIISKDNSKSQVFLKMNSLQTDDTARYYCARYYGNSPYYAIDYWGQGTSVTVSS

SEQ ID NO: 13 - protein, 113 aa, Mus musculus
DIVMTQSPSSLAVSVGEKVSMGCKSSQSLLYSSNQKNSLAWYQQKPGQSPKLLIDWASTRESGVPDRFTGSGSGTDFTLTISSVKAEDLAVYYCQQYYGYPLTFGAGTKLELK

SEQ ID NO: 14 - protein, 118 aa, Mus musculus
EVKLQESGPSLVKPSQTLSLTCSVTGYSIISDYWNWIRKFPGNKLEYLGYISYTGSTYYNPSLKSRISITRDTSKNQYYLQLNSVTTEDTATYYCARRGGWLLPFDYWGQGTTLTVSS

SEQ ID NO: 15 - protein, 106 aa, Mus musculus
DIVMTQSPAIMSASPGEKVTMTCSASSSIRYMHWYQQKPGTSPKRWISDTSKLTSGVPARFSGSGSGTSYALTISSMEAEDAATYYCHQRSSYPWTFGGGTKLEIK

SEQ ID NO: 16 - protein, 118 aa, Mus musculus
EVKLQESGPSLVKPGASVKLSCKASGYTFTSYDINWVKQRPGQGLEWIGWIFPRDNNTKYNENFKGKATLTVDTSSTTAYMELHSLTSEDSAVYFCTKENWVGDFDYWGQGTTLTLSS

SEQ ID NO: 17 - protein, 106 aa, Mus musculus
EIVLTQSPATLSLSPGERATLSCRASSSVSYIYWFQQKPGQAPRLLIYAAFNRATGIPARFSGSGSGTDYTLTISSLEPEDFAVYYCQQWSNNPLTFGQGTKVEIK

SEQ ID NO: 18 - protein, 106 aa, Mus musculus
EIVLTQSPATLSLSPGERATLSCRASSSVSYIYWFQQKPGQSPRPLIYAAFNRATGIPARFSGSGSGTDYTLTISSLEPEDFAVYYCQQWSNNPLTFGQGTKVEIK

SEQ ID NO: 19 - protein, 106 aa, Mus musculus
QIVLTQSPATLSLSPGERATLSCRASSSVSYIYWFQQKPGQSPRPLIYATFNLASGIPARFSGSGSGTSYTLTISRLEPEDFAVYYCQQWSNNPLTFGQGTKVEIK

SEQ ID NO: 20 - protein, 106 aa, Mus musculus
DIQLTQSPSSLSASVGDRVTITCRASSGVSYIYWFQQKPGKAPKLLIYAAFNLASGVPSRFSGSGSGTEYTLTISSLQPEDFATYYCQQWSNNPLTFGQGTKVEIK

SEQ ID NO: 21 - protein, 106 aa, Mus musculus
DIQLTQSPSSLSASVGDRVTITCRASSGVSYIYWFQQKPGKAPKPLIYAAFNLASGVPSRFSGSGSGTEYTLTISSLQPEDFATYYCQQWSNNPLTFGQGTKVEIK

SEQ ID NO: 22 - protein, 106 aa, Mus musculus
DIQLTQSPSILSASVGDRVTITCRASSSVSYIYWFQQKPGKAPKPLIYATFNLASGVPSRFSGSGSGTSYTLTISSLQPEDFATYYCQQWSNNPLTFGQGTKVEIK

SEQ ID NO: 23 - protein, 114 aa, Mus musculus
QVQLVQSGAEVKKPGASVKVSCKASGYTFPDYYMNWVRQAPGQGLEWMGDIDPNYGGTNYAQKFQGRVTMTRDTSISTAYMELSRLRSDDTAVYYCARGALTDWGQGTMVTVSS

SEQ ID NO: 24 - protein, 114 aa, Mus musculus
QVQLVQSGAEVKKPGASVKVSCKASGYTFPDYYMNWVRQAPGQSLEWMGDIDPNYGGTNYNQKFQGRVTMTRDTSISTAYMELSRLRSDDTAVYYCARGALTDWGQGTMVTVSS

SEQ ID NO: 25 - protein, 114 aa, Mus musculus
EVQLVQSGAEVKKPGASVKVSCKASGYTFPDYYMNWVRQAPGQSLEWMGDIDPNYGGTNYNQKFQGRVTMTVDRSSSTAYMELSRLRSDDTAVYYCARGALTDWGQGTMVTVSS

SEQ ID NO: 26 - protein, 114 aa, Mus musculus
EVQLVESGGGLVQPGRSLRLSCTASGYTFPDYYMNWVRQAPGKGLEWVGDIDPNYGGTTYAASVKGRFTISVDRSKSIAYLQMSSLKTEDTAVYYCTRGALTDWGQGTMVTVSS

SEQ ID NO: 27 - protein, 114 aa, Mus musculus
EVQLVESGGGLVQPGRSLRLSCTASGYTFPDYYMNWVRQAPGKGLEWVGDIDPNYGGTTYNASVKGRFTISVDRSKSIAYLQMSSLKTEDTAVYYCARGALTDWGQGTMVTVSS

SEQ ID NO: 28 - protein, 114 aa, Mus musculus
EVQLVESGGGLVQPGRSLRLSCTASGYTFPDYYMNWVRQAPGKGLEWVGDIDPNYGGTTYNQSVKGRFTISVDRSKSIAYLQMSSLKTEDTAVYYCARGALTDWGQGTMVTVSS

SEQ ID NO: 29 - protein, 106 aa, Mus musculus
DIQMTQFPSSLCASQGGKVTVTCKASQDINNYMAWYQHKPGKGPRLLIHYTSTLLSGIPSRFSGSGSGRDYSFSISNLEPEDIATYYCLQYDNLWTFGGGTKLEIK

SEQ ID NO: 30 - protein, 119 aa, Mus musculus
EVQLQQSGPVLVKPGASVKMSCKASGYTFTDYYMNWVKQSHGKSLEWIGNINPYNGGTTYNQKFKGKATLTVDKSSRTAYMEINSLTSEDSAVYYCARGRIYDGSLDYWGQGTALTVSS

SEQ ID NO: 31 - protein, 107 aa, Mus musculus
DIVMTQSQKFMSTSVGDRVSVTCKASQSVDTNVAWYQQKPGQSPKALIFSASYRYSGVPDRFTGSGSGTDFTLTINSVQSEDLAEYFCQQYNSYPYTFGSGTKLEIK

SEQ ID NO: 32 - protein, 116 aa, Mus musculus
QVQLQQSGAELAKPGASVRLSCKASGYTFTNYWMHWVKQRPGQGLEWIGHINPSSGFTTYNQNFKDKATLTADKSSNTAYMQLSSLTYEDSAVYFCAREDYDVDYWGQGTTLTVSS

SEQ ID NO: 33 - protein, 106 aa, Mus musculus
QIVLTQSPALMSASPGEKVTMTCSASSSVSYMYWYQQKPRSSPKPWIYLTSNLASGVPARFSGSGSGTSYSLTISSMEAEDAATYYCQQWSSNPFTFGSGTKLEIK

SEQ ID NO: 34 - protein, 122 aa, Mus musculus
EVQLVESGGGLVKPGGSLKLSCAASGFTFSDYGMHWVRQAPEKGLEWVAYISSGSYTIYYTDTVKGRFTISRDNAKNTLFLQMTSLRSEDTAMYYCARRGYGSFYEYYFDYWGQGTTLTVSS

SEQ ID NO: 35 - protein, 106 aa, Mus musculus
EIVLTQSPATLSLSPGERATLSCRASSSVSYMYWYQQKPGQAPRLLIYLASNRATGIPARFSGSGSGTDYTLTISSLEPEDFAVYYCQQWSSNPFTFGQGTKLEIK

SEQ ID NO: 36 - protein, 106 aa, Mus musculus
QIVLTQSPATLSLSPGERATLSCSASSSVSYMYWYQQKPGQAPRLLIYLTSNRATGIPARFSGSGSGTDYTLTISSLEPEDFAVYYCQQWSSNPFTFGQGTKLEIK

SEQ ID NO: 37 - protein, 106 aa, Mus musculus
DIQLTQSPSSLSASVGDRVTITCRASSSVSYMYWYQQKPGKAPKLLIYLASNLASGVPSRFSGSGSGTEYTLTISSLEPEDFATYYCQQWSSNPFTFGQGTKLEIK

SEQ ID NO: 38 - protein, 106 aa, Mus musculus
QIQLTQSPSSLSASVGDRVTITCSASSSVSYMYWYQQKPGKAPKLLIYLTSNLASGVPSRFSGSGSGTEYTLTISSLEPEDFATYYCQQWSSNPFTFGQGTKLEIK

SEQ ID NO: 39 - protein, 122 aa, Mus musculus
EVQLVESGGGLVQPGGSLRLSCAASGFTFSDYGMHWVRQAPGKGLEWVSYISSGSSTIYYADSVKGRFTISRDNAKNTLYLQMSSLRAEDTAVYYCARRGYGSFYEYYFDYWGQGTTVTVSS

SEQ ID NO: 40 - protein, 122 aa, Mus musculus
EVQLVESGGGLVQPGGSLRLSCAASGFTFSDYGMHWVRQAPGKGLEWVAYISSGSYTIYYADSVKGRFTISRDNAKNTLYLQMSSLRAEDTAVYYCARRGYGSFYEYYFDYWGQGTTVTVSS

SEQ ID NO: 41 - protein, 122 aa, Mus musculus
EVQLVESGGGLVQPGGSLRLSCAASGFTFSDYGMHWVRQAPGKGLEWVAYISSGSYTIYSADSVKGRFTISRDNAKNTLYLQMSSLRAEDTAVYYCARRGYGSFYEYYFDYWGQGTTVTVSS

SEQ ID NO: 42 - protein, 122 aa, Mus musculus
QVQLVQSGAEVKKPGASVKVSCKASGFTFSDYGMHWVRQAPGQRLEWMGYISSGSSTIYYSQKFQGRVTITRDNSASTLYMELSSLRSEDTAVYYCARRGYGSFYEYYFDYWGQGTTLTVSS

SEQ ID NO: 43 - protein, 122 aa, Mus musculus
EVQLVQSGAEVKKPGASVKVSCAASGFTFSDYGMHWVRQAPGQRLEWMGYISSGSYTIYYSQKFQGRVTITRDNSASTLYMELSSLRSEDTAVYYCARRGYGSFYEYYFDYWGQGTTLTVSS

SEQ ID NO: 44 - protein, 122 aa, Mus musculus
EVQLVQSGAEVKKPGASVKVSCAASGFTFSDYGMHWVRQAPGQRLEWVAYISSGSYTIYYSQKFQGRVTITRDNSASTLYMELSSLRSEDTAVYYCARRGYGSFYEYYFDYWGQGTTLTVSS

SEQ ID NO: 45 - protein, 106 aa, Mus musculus
QIVLSQSPAILSASPGEKVTMTCRASSSVSYIYWFQQKPGSSPKPWIYATFNLASGVPARFSGSGSGTSYSLTISRVETEDAATYYCQQWSNNPLTFGAGTKLELK

SEQ ID NO: 46 - protein, 114 aa, Mus musculus
EVQLQQSGPDLVTPGASVRISCQASGYTFPDYYMNWVKQSHGKSLEWIGDIDPNYGGTTYNQKFKGKAILTVDRSSSTAYMELRSLTSEDSAVYYCARGALTDWGQGTSLTVSS

(19) United States
(12) Patent Application Publication
(10) Pub. No.: US 2016/0031990 A1
(43) Pub. Date: Feb. 4, 2016

(54) ANTAGONISTS OF PDL-1 AND PD-1 FOR THE TREATMENT OF HPV-NEGATIVE CANCERS

(71) Applicant: Medlmmune, LLC, Gaitherburg, MD (US)

(72) Inventors: Keith STEELE, Gaitherburg, MD (US); Marlon C. Rebelatto, Gaithersburg, MD (US); John Andrew Blake-Haskins, Gaithersburg, MD (US); Paul B. Robbins, Gaithersburg, MD (US); James R. Vasselli, Gaithersburg, MD (US); Ross A. Stewart, Gaithersburg, MD (US); Ramy Ibrahim, Gaithersburg, MD (US); Aiman Shalabi, Gaithersburg, MD (US)

(21) Appl. No.: 14/724,003

(22) Filed: May 28, 2015

Related U.S. Application Data
(60) Provisional application No. 62/004,731, filed on May 29, 2014.

(51) Int. Cl. C07K 16/28

(52) U.S. Cl. CPC C07K 16/2827; C07K 2317/76

(57) ABSTRACT

Provided herein are methods of treating HPV-negative tumors comprising administering an effective amount of an antagonist of the PDL-1/PD-1 interaction (e.g., an anti-PDL-1 or anti-PD-1 antibody antigen binding fragment thereof).

### CROSS-REFERENCE TO RELATED APPLICATION

This application claims benefit under 35 U.S.C. §119(e) of U.S. Provisional Application No. 62/004,731, filed on May 29, 2014, which is incorporated by reference herein in its entirety for all purposes.

### SEQUENCE LISTING

The instant application contains a Sequence Listing which has been submitted electronically in ASCII format and is hereby incorporated by reference in its entirety. Said ASCII copy, created on May 20, 2015, is named B7H1-250US1_SL.txt and is 42,414 bytes in size.

### BACKGROUND

Cancer continues to be a major global health burden. Despite progress in the treatment of cancer, there continues to be an unmet medical need for more effective and less toxic therapies, especially for those patients with advanced disease or cancers that are resistant to existing therapeutics.

The immune system is capable of identifying tumor-associated antigens and eliminating the cancerous cells expressing them. This process of tumor immune surveillance, or tumor immunoediting, plays an important role in preventing and combating the growth of tumors, and levels of tumor-infiltrating lymphocytes, and more specifically cytotoxic T cells, have been correlated to improved prognosis in a number of cancers. Thus, enhancing the immune response may provide a means to control tumors.

Recent studies suggest that the subversion of immune pathways, termed immune checkpoints, that normally serve to temper T-cell mediated immune responses and control autoimmunity, provide a common mechanism by which tumors are able evade host immune responses. Consequently, much attention has been directed to understanding immune checkpoint pathways with the hope of translating this understanding into the next generation of immunostimulatory drugs. One T-cell inhibitory checkpoint pathway signals through programmed death-1 (PD-1, CD279) and its ligand programmed death ligand-1 (PDL-1, CD274, B7-H1).

The PD-1/PDL-1 pathway is believed to primarily function to limit autoimmunity by restraining the activity of T-cells in the periphery during chronic inflammation, infection and cancer. This pathway is thought to deliver inhibitory signals that predominantly regulate the effector phase of T-cells against tumor cells and has been implicated in tumor growth and progression.

PD-1 is expressed on activated T-cells and regulatory T cells, NK-T cells, B-cells, and activated monocytes. In normal tissue, PDL-1 is expressed on T-cells, B-cells, dendritic cells, macrophages, mesenchymal stem cells, bone marrow-derived mast cells, as well as various nonhematopoietic cells. PDL-1 is also expressed by tumors and acts at multiple sites to help tumors evade detection and elimination by the host immune system. PDL-1 is expressed in a broad range of cancers with a high frequency. In some cancers, expression of PDL-1 has been associated with reduced survival and unfavorable prognosis.

Antibodies that block the interaction between PD-1 and PDL-1 are able to relieve PDL-1-dependent immunosuppressive effects and enhance the cytotoxic activity of anti-tumor T-cells in vitro and some of these antibodies (e.g., MEDI4736) are being investigated as cancer treatments.

Several types of cancer are associated with human papilloma virus (HPV), and the expression of PD-1 has been shown to be upregulated on tumor infiltrating lymphocytes isolated from patients with HPV-associated cancers. In addition, the expression of PDL-1 has been shown to be increased in HPV-associated cancers. See e.g. Pike S. L. et al., Cancer Research, 73: 1733 (20130; Pai S. I, OncoImmunology, 2(5):e24065-1 (2013).

The efficacy of several antibody therapeutics has been shown to be correlated with antigen expression level. For example, Herceptin® (trastuzumab) binds to HER2 protein, and data from efficacy trials with Herceptin®shows that beneficial treatment effects were largely limited to patients with the highest levels of HER2 protein expression. The degree of HER2 overexpression is considered a predictor of treatment effect, and Herceptin® is specifically indicated for cancers overexpressing HER2.

Thus, given the high unmet need to treating cancers, the ability of PD-1 antagonists (e.g., antibodies that block the interaction of PD-1 and PDL-1) to treat HPV-positive and HPV-negative cancers was investigated to determine if HPV-positive tumor status was a predictor of treatment efficacy.

### BRIEF SUMMARY

Methods of treating HPV-negative cancers are provided herein.

In some instances, a method of treating cancer comprises administering a PDL-1 antagonist to a human patient having cancer, wherein the cancer is HPV-negative. In some instances, the PDL-1 antagonist is an anti-PDL-1 antibody or antigen-binding fragment thereof. In some instances, the PDL-1 antagonist (e.g., an anti-PDL-1 antibody or antigen-binding fragment thereof) inhibits the interaction of PDL-1 and PD-1. In some instances, the PDL-1 antagonist (e.g., an anti-PDL-1 antibody or antigen-binding fragment thereof) increases an immune response to an HPV-negative cancer.

In some instances, a method of treating cancer comprises administering a PD-1 antagonist to a human patient having cancer, wherein the cancer is HPV-negative. In some instances, the PD-1 antagonist is an anti-PD-1 antibody or antigen-binding fragment thereof. In some instances, the PD-1 antagonist (e.g., an anti-PD-1 antibody or antigen-binding fragment thereof) inhibits the interaction of PDL-1 and PD-1. In some instances, the PD-1 antagonist (e.g., an anti-PD-1 antibody or antigen-binding fragment thereof) increases an immune response to an HPV-negative cancer.

In some instances, a method of treating cancer comprises administering an antagonist of the interaction of PDL-1 and PD-1 to a human patient having cancer, wherein the cancer is HPV-negative.

In some instances, the antagonist is MEDI4736 or an antigen-binding fragment thereof.

In some instances, the method further comprises determining if the cancer is HPV-negative.

In some instances, the administration reduces tumor growth. In some instances, the administration decreases tumor size. In some instances, the administration decreases tumor size by at least 25%. In some instances, the administration decreases tumor size by at least 25% within about 12 weeks of the first administration of the antagonist.

In some instances, the administration produces an AUC (tau) of about 100 to about 2,500 d·μg/mL. In some instances, the administration produces a Cmax of about 15 to about 350 μg/mL.

In some instances, the half-life of the MEDI4736 or the antigen-binding fragment thereof is about 5 to about 25 days. In some instances, the clearance of the MEDI4736 or the antigen-binding fragment thereof is about 1-10 ml/day/kg.

In some instances, about 0.1, about 0.3, about 1, about 3, about 10, or about 15 mg/kg MEDI4736 or an antigen-binding fragment thereof is administered. In some instances, about 0.1 mg/kg MEDI4736 or an antigen-binding fragment thereof is administered. In some instances, about 0.3 mg/kg MEDI4736 or an antigen-binding fragment thereof is administered. In some instances, about 1 mg/kg MEDI4736 or an antigen-binding fragment thereof is administered. In some instances, about 3 mg/kg MEDI4736 or an antigen-binding fragment thereof is administered. In some instances, about 10 mg/kg MEDI4736 or an antigen-binding fragment thereof is administered. In some instances, about 15 mg/kg MEDI4736 or an antigen-binding fragment thereof is administered.

In some instances, the administration is repeated about every 14 to 21 days. In some instances, the administration is repeated about every 14 days.

In some instances, the tumor size decreases or tumor growth is reduced and MEDI4736 or an antigen-binding fragment thereof is subsequently administered as a maintenance therapy about every 2 months.

In some instances, the administration results in a partial response. In some instances, the administration results in a complete response.

In some instances, the cancer squamous cell carcinoma of the head and neck (SCCHN). In some instances, the cancer is oropharyngeal squamous cell carcinoma.

In some instances, the tumor is refractory to at least one chemotherapeutic agent.

### BRIEF DESCRIPTION OF THE DRAWINGS/FIGURES

FIG. 1 shows the timeline of treatment with MEDI4736 administered intravenously (IV) every two weeks (Q2W) Immune-related response criteria (irRC) are measured after weeks 6, 12, and 16 and then every 8 weeks.

FIG. 2A shows the study flow diagram for the dose-expansion and dose-escalation portions of the study. The dose expansion portion of the study is conducted using a two-week dosing schedule (Q2W) and a three-week dosing schedule (Q3W). Patients with non-small cell lung cancer (NSCLC), melanoma, and other tumors are evaluated in the escalation portion of the study; 2B shows the tumor types in the expansion.

FIG. 3 shows a summary of the pharmacokinetic data obtained after administering MEDI4736 (Q2W) at 0.1 mg/kg or 0.3 mg/kg during the dose-escalation phase of the study. “AUC”=area under the curve; “Cmax”=maximum observed concentration.

FIG. 4 shows the concentration of MEDI4736 over time that was observed in patients receiving 0.1 mg/kg, 0.3 mg/kg, or 1 mg/kg MEDI4736 (Q2W) during the dose-escalation phase of the study.

FIG. 5 shows the target engagement over time that was observed in patients receiving 0.1 mg/kg, 0.3 mg/kg, or 1 mg/kg MEDI4736 (Q2W) during the dose-escalation phase of the study. “LLOQ”=lower limit of quantitation.

FIG. 6 shows the clinical activity of MEDI4736 observed in patients with non-small cell lung cancer (NSCLC), melanoma, or colorectal cancer (CRC) receiving 0.1 mg/kg, 0.3 mg/kg, or 1 mg/kg MEDI4736. Best responses are characterized as stable disease (SD), progressive disease (PD), partial response (PR), or not evaluable (NE)

FIG. 7 shows the effect of MEDI4736 on tumor size in patients receiving 0.1 mg/kg, 0.3 mg/kg, 1 mg/kg, 10 mg/kg or 15 mg/kg MEDI4736.

FIG. 8 shows effect of 10 mg/kg MEDI4736 on NSCLC tumors.

FIGS. 9A and 9B show the effect of 10 mg/kg on in HPV-positive (#) and HPV-negative squamous cell carcinoma of the head and neck (SCCHN) tumors. 9A shows the change from baseline over time, and 9B shows the best change in baseline observed in each patient at any time point.

FIG. 10 shows the results of subjects treated with MEDI4736 with 24 months of follow-up. Response rates are presented based on HPV status and/or PDL1 status.

### DETAILED DESCRIPTION

Provided herein are methods for treating HPV-negative cancers. The methods provided include administering an effective amount of one or more antagonists of the interaction of PD-1 with PDL-1.

### I. Definitions

It is to be noted that the term “a” or “an” entity refers to one or more of that entity; for example, “an anti-PDL-1 antibody” is understood to represent one or more anti-PDL-1 antibodies. As such, the terms “a” (or “an”), “one or more,” and “at least one” can be used interchangeably herein.

The terms “inhibit,” “block,” and “suppress” are used interchangeably herein and refer to any statistically significant decrease in biological activity, including full blocking of the activity. For example, “inhibition” can refer to a decrease of at least 10%, or at least 20%, or at least 30%, or at least 40%, or at least 50%, or at least 60%, or at least 70%, or at least 80%, or at least 90%, or about 100% in biological activity. Accordingly, when the terms “inhibition” or “suppression” are applied to describe for example, an effect on PD-1 and/or PDL-1 expression on T-cells and/or T-cell-mediated cytolytic activity, the term refers to for example, the ability of an antagonist such as, an anti-PD-1 antibody and/or anti-PDL1 antibody, to statistically significantly decrease the activity of the antigen to which the antagonist binds. For example the term inhibit or block may be used to refer to the ability of an anti-PDL-1 antibody and/or an anti-PD1 antibody to decreased the expression of PDL-1 or PD1 and/or the ability of the antibody to increase T cell-mediated cytolytic activity in vitro or in vivo, relative to expression and/or T cell-medicated cytolytic activity in an untreated cell population (control). The term inhibit or block is also used herein to refer to the ability of an antagonist (e.g., anti-PDL-1 or anti-PD1 antibody or antigen-binding fragment thereof) to decrease the ability of PDL-1 to interact with (i.e., bind to) PD-1.

The term “inhibit activation” or “suppress activation” of an effector cell such as a T cell as used herein, refers to the ability of a composition disclosed herein such as, an anti-PD1 antibody and/or an anti-PDL-1 antibody to statistically significantly decrease the activation of an effector cell expressing the surface antigen (e.g., a T cell) relative to the activation of the effector cell in the absence of the antagonist antibody. In one embodiment, the activation of a T cell or other effector cell expressing the surface antigen is decreased by at least 10%, or at least 20%, or at least 30%, or at least 40%, or at least 50%, or at least 60%, or at least 70%, or at least 80%, or at least 90%, or about 100% when cells are contacted with the antagonist antibody, relative to the activation measured in the absence of the antagonist antibody.

Effector cell activation can be assayed using techniques known in the art that measure for example, surface marker expression, intracellular signaling, rates of cell division, cytolytic activity and/or cytokine production.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc. to form Antibody Drug Conjugates (ADC).

The terms “antibody” or “immunoglobulin,” are used interchangeably herein, and include whole antibodies and any antigen binding fragment or single chains thereof. A typical antibody comprises at least two heavy (H) chains and two light (L) chains interconnected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed Complementarity Determining Regions (CDR), interspersed with regions that are more conserved, termed framework regions (FW). Each VH and VL is composed of three CDRs and four FWs, arranged from amino-terminus to carboxy-terminus in the following order: FW1, CDR1, FW2, CDR2, FW3, CDR3, FW4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. Exemplary antibodies of the present disclosure include typical antibodies, scFvs, and combinations thereof where, for example, an scFv is covalently linked (for example, via peptidic bonds or via a chemical linker) to the N-terminus of either the heavy chain and/or the light chain of a typical antibody, or intercalated in the heavy chain and/or the light chain of a typical antibody. Additional exemplary “antibodies” herein include fusion proteins comprising an antibody portion, and any other modified immunoglobulin molecule comprising an antigen recognition site. For the purposes of this disclosure, the term antibody also encompasses Fc fusion proteins containing immunoglobulin-derived, naturally occurring and/or synthetic amino acid sequences (e.g., peptibodies) that bind an expressed on a cell of interest to be targeted (e.g., cell surface immune checkpoint antigen such as PD-1L.)

The phrase “antigen binding fragment” refers to a portion of an intact antibody and/or refers to the antigenic determining variable regions of an intact antibody. It is known that the antigen binding function of an antibody can be performed by fragments of a full-length antibody. Examples of antibody fragments include, but are not limited to, Fab, Fab′, F(ab′)2, and Fv fragments, linear antibodies, single chain antibodies, diabodies, and multispecific antibodies formed from antibody fragments.

In particular embodiments, the antibodies used according to the disclosed methods have reduced effector function. In some embodiments, the antibodies contain mutations in the Fc region responsible for effector function, such as, one or more mutations described in Int. Appl. Publ. Nos. WO09/100309, WO06/076594, WO06/053301, WO06/047350; and WO99/58572; U.S. Pat. Nos. 6,737,056 and 5,624,821, and U.S. Appl. Publ. Nos. US 2010/0166740 and 2006/0134709, the contents of each of which is herein incorporated by reference in its entirety. By “reduced effector function” is intended a reduction of a specific effector function such as, ADCC or CDC, in comparison to a control (for example a polypeptide with a wildtype Fc region), by at least 20%, at least 30% or by at least 50%.

A “blocking” antibody or an “antagonist” antibody or agent is one which inhibits or reduces biological activity of the antigen it binds, e.g., inhibiting or reducing the ability of PDL-1 to interact with or bind to PD-1. In a certain embodiment blocking antibodies or antagonist antibodies substantially or completely inhibit the biological activity of the antigen. Desirably, the biological activity is reduced by at least 10%, 20%, 30%, 50%, 70%, 80%, 90%, 95%, or about 100%.

As used herein, the term “specifically binds” refers to the situation in which one member of a specific binding pair, such as an antibody, does not significantly bind to molecules other than its specific binding partner(s) (i.e., cross-reactivity of less than about 25%, 20%, 15%, 10%, or 5%) as measured by a technique in the art, at a diagnostically or therapeutically relevant concentration e.g., by competition ELISA or by measurement of KD with BIACORE or KINEXA assay.

As used herein, the term “MEDI4736” refers to an antibody having a light chain variable region comprising the amino acid sequence of SEQ ID NO:1 and a heavy chain variable region comprising the amino acid sequence of SEQ ID NO:2. MEDI4736 is further disclosed in Intl. Appl. Publ. No. WO 2011/066389 A1 and U.S. Appl. Publ. No. 2010/0028330, the disclosure of each of which is herein incorporated by reference in its entirety. The Fc domain of MEDI4736 contains a triple mutation in the constant domain of the IgG1 heavy chain that reduces binding to the complement component C1q and the Fcγ receptors responsible for mediating antibody-dependent cell-mediated cytotoxicity (ADCC). MEDI4736 specifically binds PDL-1 and blocks the binding of PDL-1 to the PD-1 and CD80 (B7.1) receptors. MEDI4736 can relieve PDL-1-mediated suppression of human T-cell activation in vitro and inhibits tumor growth in a xenograft model via a T-cell dependent mechanism.

MEDI4736 and antigen-binding fragments thereof for use in the methods provided herein comprises a heavy chain and a light chain or a heavy chain variable region and a light chain variable region. In a specific embodiment, MEDI4736 or an antigen-binding fragment thereof for use in the methods provided herein comprises a light chain variable region comprising the amino acid sequence of SEQ ID NO:1 and a heavy chain variable region comprising the amino acid sequence of SEQ ID NO:2. In a particular embodiment, MEDI4736 or an antigen-binding fragment thereof for use in the methods provided herein comprises a heavy chain variable region and a light chain variable region, wherein the heavy chain variable region comprises the CDR1, CDR2, and CDR3 sequences of SEQ ID NOS:3, 4, and 5, respectively, and wherein the light chain variable region comprises the CDR1, CDR2, and CDR3 sequences of SEQ ID NOS:6, 7, and 8, respectively. Those of ordinary skill in the art would easily be able to identify Chothia-defined, Abm-defined or other CDR definitions known to those of ordinary skill in the art. In a specific embodiment, MEDI4736 or an antigen-binding fragment thereof for use in the methods provided herein comprises the variable heavy chain and variable light chain CDR sequences of the 2.14H9OPT antibody as disclosed in Intl. Appl. Publ. No. WO 2011/066389, the contents of which are herein incorporated by reference in its entirety.

The term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

The term “pharmaceutical composition” refers to a preparation which is in such form as to permit the biological activity of the active ingredient to be effective, and which contains no additional components which are unacceptably toxic to a subject to which the composition would be administered. Such composition can be sterile.

Terms such as “treating” or “treatment” or “to treat” refer to therapeutic measures that cure, slow down, lessen symptoms of, and/or halt progression of a diagnosed pathologic condition or disorder. Thus, those in need of treatment include those already diagnosed with or suspected of having the disorder. Prophylactic or preventative measures refer to measures that prevent and/or slow the development of a targeted pathologic condition or disorder. Thus, those in need of prophylactic or preventative measures include those prone to have the disorder and those in whom the disorder is to be prevented.

### II. Antagonists

Interaction of PDL-1 and PD-1 has been found to provide a crucial negative co-stimulatory signal to T and B cells. The methods described herein provide methods of administering antagonists of the PDL-1/PD-1 interaction to treat HPV-negative cancers. Antagonists of the interaction of PDL-1 and PD-1 are antagonists that specifically bind to PDL-1 and/or PD-1 and inhibit the ability of PDL-1 to interact with or bind to PD-1 (i.e., the ability of PD-1 to interact with or bind to PDL-1).

Antagonists that specifically bind PD-1 or PDL-1 and inhibit their interaction are known and/or can be readily identified and prepared using techniques known in the art. In some embodiments, the antagonist of PDL-1 and/or PD-1 increases immune responses to HPV-negative cancers. In some embodiments, the antagonist of the PDL-1/PD-1 interaction is an antibody or an antigen-binding fragment thereof that specifically binds PD-1 and/or PDL-1. Methods of confirming that an antagonist can inhibit the interaction of PDL-1 and PD-1 are known. For example, certain assays that can be used to demonstrate that an antagonist can inhibit the interaction of PDL-1 and PD-1 are disclosed in WO 2012/145493, which is herein incorporated by reference in its entirety.

The methods described herein also provide methods of administering PD-1 antagonists to treat HPV-negative cancers. In some embodiments, the PD-1 antagonist inhibits the interaction of PDL-1 and PD-1. In some embodiments, the PD-1 antagonist is an antibody or an antigen-binding fragment thereof that binds PD-1. In additional embodiments, the PD-1 antagonist is an Fc fusion protein comprising an IgG Fc region fused to one or more polypeptides such as a portion of PDL-1, an scFv, or a synthetic peptide that binds PD-1. Certain PD-1 antagonists are disclosed, for example, in WO 2012/145493.

In some embodiments, the PD-1 antagonist competes with an antibody containing a VL having the sequence recited in SEQ ID NO:29 and a VH having the sequence recited in SEQ ID NO:30 for binding to PD-1. In additional embodiments, the PD-1 antagonist binds to the same epitope of PD-1 as an antibody containing a VL having the sequence recited in SEQ ID NO:29 and a VH having the sequence recited in SEQ ID NO:30. In additional embodiments, the PD-1 antagonists comprises a VL having the sequence recited in SEQ ID NO:29 and a VH having the sequence recited in SEQ ID NO:30.

In some embodiments, the PD-1 antagonist competes with an antibody containing a VL having the sequence recited in SEQ ID NO:31 and a VH having the sequence recited in SEQ ID NO:32 for binding to PD-1. In additional embodiments, the PD-1 antagonist binds to the same epitope of PD-1 as an antibody containing a VL having the sequence recited in SEQ ID NO:31 and a VH having the sequence recited in SEQ ID NO:32. In additional embodiments, the PD-1 antagonists comprises a VL having the sequence recited in SEQ ID NO:31 and a VH having the sequence recited in SEQ ID NO:32.

In some embodiments, the PD-1 antagonist competes with an antibody containing a VL having the sequence recited in SEQ ID NO:33 and a VH having the sequence recited in SEQ ID NO:34 for binding to PD-1. In additional embodiments, the PD-1 antagonist binds to the same epitope of PD-1 as an antibody containing a VL having the sequence recited in SEQ ID NO:33 and a VH having the sequence recited in SEQ ID NO:34. In additional embodiments, the PD-1 antagonists comprises a VL having the sequence recited in SEQ ID NO:33 and a VH having the sequence recited in SEQ ID NO:34.

In some embodiments, the PD-1 antagonist competes with an antibody containing a VL having the sequence recited in any one of SEQ ID NOS: 35-38 and a VH having the sequence recited in any one of SEQ ID NOS:39-44 for binding to PD-1. In additional embodiments, the PD-1 antagonist binds to the same epitope of PD-1 as an antibody containing a VL having the sequence recited in any one of SEQ ID NOS:35-38 and a VH having the sequence recited in any one of SEQ ID NOS:39-44. In some embodiments, the PD-1 antagonist comprises a VL having the sequence recited in any one of SEQ ID NOS: 35-38 and a VH having the sequence recited in any one of SEQ ID NOS:39-44.

In some embodiments, the PD-1 antagonist comprises a VL having the sequence recited in SEQ ID NO:35 and a VH having the sequence recited in any one of SEQ ID NOS:39-44. In additional embodiments, the PD-1 antagonists comprises a VL having the sequence recited in SEQ ID NO:35 and a VH having the sequence recited in SEQ ID NO:39. In additional embodiments, the PD-1 antagonists comprises a VL having the sequence recited in SEQ ID NO:35 and a VH having the sequence recited in SEQ ID NO:40. In additional embodiments, the PD-1 antagonists comprises a VL having the sequence recited in SEQ ID NO:35 and a VH having the sequence recited in SEQ ID NO:41. In additional embodiments, the PD-1 antagonists comprises a VL having the sequence recited in SEQ ID NO:35 and a VH having the sequence recited in SEQ ID NO:42. In additional embodiments, the PD-1 antagonists comprises a VL having the sequence recited in SEQ ID NO:35 and a VH having the sequence recited in SEQ ID NO:43. In additional embodiments, the PD-1 antagonists comprises a VL having the sequence recited in SEQ ID NO:35 and a VH having the sequence recited in SEQ ID NO:44.

In some embodiments, the PD-1 antagonist comprises a VL having the sequence recited in SEQ ID NO:36 and a VH having the sequence recited in any one of SEQ ID NOS:39-44. In additional embodiments, the PD-1 antagonists comprises a VL having the sequence recited in SEQ ID NO:36 and a VH having the sequence recited in SEQ ID NO:39. In additional embodiments, the PD-1 antagonists comprises a VL having the sequence recited in SEQ ID NO:36 and a VH having the sequence recited in SEQ ID NO:40. In additional embodiments, the PD-1 antagonists comprises a VL having the sequence recited in SEQ ID NO:36 and a VH having the sequence recited in SEQ ID NO:41. In additional embodiments, the PD-1 antagonists comprises a VL having the sequence recited in SEQ ID NO:36 and a VH having the sequence recited in SEQ ID NO:42. In additional embodiments, the PD-1 antagonists comprises a VL having the sequence recited in SEQ ID NO:36 and a VH having the sequence recited in SEQ ID NO:43. In additional embodiments, the PD-1 antagonists comprises a VL having the sequence recited in SEQ ID NO:36 and a VH having the sequence recited in SEQ ID NO:44.

In some embodiments, the PD-1 antagonist comprises a VL having the sequence recited in SEQ ID NO:37 and a VH having the sequence recited in any one of SEQ ID NOS:39-44. In additional embodiments, the PD-1 antagonists comprises a VL having the sequence recited in SEQ ID NO:37 and a VH having the sequence recited in SEQ ID NO:39. In additional embodiments, the PD-1 antagonists comprises a VL having the sequence recited in SEQ ID NO:37 and a VH having the sequence recited in SEQ ID NO:40. In additional embodiments, the PD-1 antagonists comprises a VL having the sequence recited in SEQ ID NO:37 and a VH having the sequence recited in SEQ ID NO:41. In additional embodiments, the PD-1 antagonists comprises a VL having the sequence recited in SEQ ID NO:37 and a VH having the sequence recited in SEQ ID NO:42. In additional embodiments, the PD-1 antagonists comprises a VL having the sequence recited in SEQ ID NO:37 and a VH having the sequence recited in SEQ ID NO:43. In additional embodiments, the PD-1 antagonists comprises a VL having the sequence recited in SEQ ID NO:37 and a VH having the sequence recited in SEQ ID NO:44.

In some embodiments, the PD-1 antagonist comprises a VL having the sequence recited in SEQ ID NO:38 and a VH having the sequence recited in any one of SEQ ID NOS:39-44. In additional embodiments, the PD-1 antagonists comprises a VL having the sequence recited in SEQ ID NO:38 and a VH having the sequence recited in SEQ ID NO:39. In additional embodiments, the PD-1 antagonists comprises a VL having the sequence recited in SEQ ID NO:38 and a VH having the sequence recited in SEQ ID NO:40. In additional embodiments, the PD-1 antagonists comprises a VL having the sequence recited in SEQ ID NO:38 and a VH having the sequence recited in SEQ ID NO:41. In additional embodiments, the PD-1 antagonists comprises a VL having the sequence recited in SEQ ID NO:38 and a VH having the sequence recited in SEQ ID NO:42. In additional embodiments, the PD-1 antagonists comprises a VL having the sequence recited in SEQ ID NO:38 and a VH having the sequence recited in SEQ ID NO:43. In additional embodiments, the PD-1 antagonists comprises a VL having the sequence recited in SEQ ID NO:38 and a VH having the sequence recited in SEQ ID NO:44.

In some embodiments, the PD-1 antagonist comprises a VL having the sequence recited in any one of SEQ ID NOS: 35-38 and a VH having the sequence recited in SEQ ID NO:39. In some embodiments, the PD-1 antagonist comprises a VL having the sequence recited in any one of SEQ ID NOS: 35-38 and a VH having the sequence recited in SEQ ID NO:40. In some embodiments, the PD-1 antagonist comprises a VL having the sequence recited in any one of SEQ ID NOS: 35-38 and a VH having the sequence recited in SEQ ID NO:41. In some embodiments, the PD-1 antagonist comprises a VL having the sequence recited in any one of SEQ ID NOS: 35-38 and a VH having the sequence recited in SEQ ID NO:42. In some embodiments, the PD-1 antagonist comprises a VL having the sequence recited in any one of SEQ ID NOS: 35-38 and a VH having the sequence recited in SEQ ID NO:43. In some embodiments, the PD-1 antagonist comprises a VL having the sequence recited in any one of SEQ ID NOS: 35-38 and a VH having the sequence recited in SEQ ID NO:44.

In additional embodiments, the PD-1 antagonist competes with nivolumab (e.g., BMS-936558/MDX-1106/ONO-4538) for binding to PD-1. In other embodiments, the PD-1 antagonist binds to the same epitope of PD-1 as nivolumab. In particular embodiments, the PD-1 antagonist used according to the disclosed methods is nivolumab. See, e.g., Brahmer et al., J. Clin. Oncol. 28:3167-3175 (2010) and Topalian et al., N. Engl. J. Med. 28:366 (26):2443-54 (2012).

In some embodiments, the PD-1 antagonist competes with pidilizumab (e.g., CT-011; Curetech/Teva) for binding to PD-1. In additional embodiments, the PD-1 antagonist binds to the same epitope of PD-1 as pidilizumab. In particular embodiments, the PD-1 antagonist used according to the disclosed methods is pidilizumab. See, e.g., Berger et al., Clin. Cancer Res. 14:3044-3051 (2008).

In some embodiments, the PD-1 antagonist competes with lambrolizumab (e.g., MK-3475; Merck) for binding to PD-1. In additional embodiments, the PD-1 antagonist binds to the same epitope of PD-1 as lambrolizumab. In particular embodiments, the PD-1 antagonist used according to the disclosed methods is lambrolizumab. See, e.g., Hamid et al., N. Engl. J. Med. 11369(2):134-44 (2013).

The methods described herein also provide methods of administering PDL-1 antagonists to treat HPV-negative cancers. In some embodiments, the PDL-1 antagonist inhibits the interaction of PDL-1 and PD-1. In some embodiments, the PDL-1 antagonist is an antibody or an antigen-binding fragment thereof that binds to PDL-1. In additional embodiments, the PDL-1 antagonist is an Fc fusion protein comprising an IgG Fc region fused to one or more polypeptides such as a portion of PD-1, an scFv, or a synthetic peptide that binds PDL-1.

In some embodiments, the PDL-1 antagonist competes with MEDI4736 (MedImmune/AstraZeneca) for binding to PDL-1. In additional embodiments, the PDL-1 antagonist binds to the same epitope of PDL-1 as MEDI4736. In particular embodiments, the PDL-1 antagonist used according to the disclosed methods is MEDI4736.

Certain other PDL-1 antagonists are disclosed, for example, in WO 2012/145493.

In some embodiments, the PDL-1 antagonist competes with an antibody containing a VL having the sequence recited in SEQ ID NO:9 and a VH having the sequence recited in SEQ ID NO:10 for binding to PDL-1. In additional embodiments, the PDL-1 antagonist binds to the same epitope of PDL-1 as an antibody containing a VL having the sequence recited in SEQ ID NO:9 and a VH having the sequence recited in SEQ ID NO:10. In additional embodiments, the PDL-1 antagonists comprises a VL having the sequence recited in SEQ ID NO:9 and a VH having the sequence recited in SEQ ID NO:10.

In some embodiments, the PDL-1 antagonist competes with an antibody containing a VL having the sequence recited in SEQ ID NO:11 and a VH having the sequence recited in SEQ ID NO:12 for binding to PDL-1. In additional embodiments, the PDL-1 antagonist binds to the same epitope of PDL-1 as an antibody containing a VL having the sequence recited in SEQ ID NO:11 and a VH having the sequence recited in SEQ ID NO:12. In additional embodiments, the PDL-1 antagonists comprises a VL having the sequence recited in SEQ ID NO:11 and a VH having the sequence recited in SEQ ID NO:12.

In some embodiments, the PDL-1 antagonist competes with an antibody containing a VL having the sequence recited in SEQ ID NO:13 and a VH having the sequence recited in SEQ ID NO:14 for binding to PDL-1. In additional embodiments, the PDL-1 antagonist binds to the same epitope of PDL-1 as an antibody containing a VL having the sequence recited in SEQ ID NO:13 and a VH having the sequence recited in SEQ ID NO:14. In additional embodiments, the PDL-1 antagonists comprises a VL having the sequence recited in SEQ ID NO:13 and a VH having the sequence recited in SEQ ID NO:14.

In some embodiments, the PDL-1 antagonist competes with an antibody containing a VL having the sequence recited in SEQ ID NO:15 and a VH having the sequence recited in SEQ ID NO:16 for binding to PDL-1. In additional embodiments, the PDL-1 antagonist binds to the same epitope of PDL-1 as an antibody containing a VL having the sequence recited in SEQ ID NO:15 and a VH having the sequence recited in SEQ ID NO:16. In additional embodiments, the PDL-1 antagonists comprises a VL having the sequence recited in SEQ ID NO:15 and a VH having the sequence recited in SEQ ID NO:16.

In some embodiments, the PDL-1 antagonist competes with an antibody containing a VL having the sequence recited in SEQ ID NO:45 and a VH having the sequence recited in SEQ ID NO:46 for binding to PDL-1. In additional embodiments, the PDL-1 antagonist binds to the same epitope of PDL-1 as an antibody containing a VL having the sequence recited in SEQ ID NO:45 and a VH having the sequence recited in SEQ ID NO:46. In additional embodiments, the PDL-1 antagonists comprises a VL having the sequence recited in SEQ ID NO:45 and a VH having the sequence recited in SEQ ID NO:46.

In some embodiments, the PDL-1 antagonist competes with an antibody containing a VL having the sequence recited in any one of SEQ ID NOS:17-22 and a VH having the sequence recited in any one of SEQ ID NOS:23-28 for binding to PDL-1. In additional embodiments, the PDL-1 antagonist binds to the same epitope of PDL-1 as an antibody containing a VL having the sequence recited in any one of SEQ ID NOS:17-22 and a VH having the sequence recited in any one of SEQ ID NOS:23-28. In some embodiments, the PDL-1 antagonist comprises a VL having the sequence recited in any one of SEQ ID NOS:17-22 and a VH having the sequence recited in any one of SEQ ID NOS:23-28.

In some embodiments, the PDL-1 antagonist comprises a VL having the sequence recited in SEQ ID NO:17 and a VH having the sequence recited in any one of SEQ ID NOS:23-28. In some embodiments, the PDL-1 antagonist comprises a VL having the sequence recited in SEQ ID NO:17 and a VH having the sequence recited in SEQ ID NO:23. In some embodiments, the PDL-1 antagonist comprises a VL having the sequence recited in SEQ ID NO:17 and a VH having the sequence recited in SEQ ID NO:24. In some embodiments, the PDL-1 antagonist comprises a VL having the sequence recited in SEQ ID NO:17 and a VH having the sequence recited in SEQ ID NO:25. In some embodiments, the PDL-1 antagonist comprises a VL having the sequence recited in SEQ ID NO:17 and a VH having the sequence recited in SEQ ID NO:26. In some embodiments, the PDL-1 antagonist comprises a VL having the sequence recited in SEQ ID NO:17 and a VH having the sequence recited in SEQ ID NO:27. In some embodiments, the PDL-1 antagonist comprises a VL having the sequence recited in SEQ ID NO:17 and a VH having the sequence recited in SEQ ID NO:28.

In some embodiments, the PDL-1 antagonist comprises a VL having the sequence recited in SEQ ID NO:18 and a VH having the sequence recited in any one of SEQ ID NOS:23-28. In some embodiments, the PDL-1 antagonist comprises a VL having the sequence recited in SEQ ID NO:18 and a VH having the sequence recited in SEQ ID NO:23. In some embodiments, the PDL-1 antagonist comprises a VL having the sequence recited in SEQ ID NO:18 and a VH having the sequence recited in SEQ ID NO:24. In some embodiments, the PDL-1 antagonist comprises a VL having the sequence recited in SEQ ID NO:18 and a VH having the sequence recited in SEQ ID NO:25. In some embodiments, the PDL-1 antagonist comprises a VL having the sequence recited in SEQ ID NO:18 and a VH having the sequence recited in SEQ ID NO:26. In some embodiments, the PDL-1 antagonist comprises a VL having the sequence recited in SEQ ID NO:18 and a VH having the sequence recited in SEQ ID NO:27. In some embodiments, the PDL-1 antagonist comprises a VL having the sequence recited in SEQ ID NO:18 and a VH having the sequence recited in SEQ ID NO:28.

In some embodiments, the PDL-1 antagonist comprises a VL having the sequence recited in SEQ ID NO:19 and a VH having the sequence recited in any one of SEQ ID NOS:23-28. In some embodiments, the PDL-1 antagonist comprises a VL having the sequence recited in SEQ ID NO:19 and a VH having the sequence recited in SEQ ID NO:23. In some embodiments, the PDL-1 antagonist comprises a VL having the sequence recited in SEQ ID NO:19 and a VH having the sequence recited in SEQ ID NO:24. In some embodiments, the PDL-1 antagonist comprises a VL having the sequence recited in SEQ ID NO:19 and a VH having the sequence recited in SEQ ID NO:25. In some embodiments, the PDL-1 antagonist comprises a VL having the sequence recited in SEQ ID NO:19 and a VH having the sequence recited in SEQ ID NO:26. In some embodiments, the PDL-1 antagonist comprises a VL having the sequence recited in SEQ ID NO:19 and a VH having the sequence recited in SEQ ID NO:27. In some embodiments, the PDL-1 antagonist comprises a VL having the sequence recited in SEQ ID NO:19 and a VH having the sequence recited in SEQ ID NO:28.

In some embodiments, the PDL-1 antagonist comprises a VL having the sequence recited in SEQ ID NO:20 and a VH having the sequence recited in any one of SEQ ID NOS:23-28. In some embodiments, the PDL-1 antagonist comprises a VL having the sequence recited in SEQ ID NO:20 and a VH having the sequence recited in SEQ ID NO:23. In some embodiments, the PDL-1 antagonist comprises a VL having the sequence recited in SEQ ID NO:20 and a VH having the sequence recited in SEQ ID NO:24. In some embodiments, the PDL-1 antagonist comprises a VL having the sequence recited in SEQ ID NO:20 and a VH having the sequence recited in SEQ ID NO:25. In some embodiments, the PDL-1 antagonist comprises a VL having the sequence recited in SEQ ID NO:20 and a VH having the sequence recited in SEQ ID NO:26. In some embodiments, the PDL-1 antagonist comprises a VL having the sequence recited in SEQ ID NO:20 and a VH having the sequence recited in SEQ ID NO:27. In some embodiments, the PDL-1 antagonist comprises a VL having the sequence recited in SEQ ID NO:20 and a VH having the sequence recited in SEQ ID NO:28.

In some embodiments, the PDL-1 antagonist comprises a VL having the sequence recited in SEQ ID NO:21 and a VH having the sequence recited in any one of SEQ ID NOS:23-28. In some embodiments, the PDL-1 antagonist comprises a VL having the sequence recited in SEQ ID NO:21 and a VH having the sequence recited in SEQ ID NO:23. In some embodiments, the PDL-1 antagonist comprises a VL having the sequence recited in SEQ ID NO:21 and a VH having the sequence recited in SEQ ID NO:24. In some embodiments, the PDL-1 antagonist comprises a VL having the sequence recited in SEQ ID NO:21 and a VH having the sequence recited in SEQ ID NO:25. In some embodiments, the PDL-1 antagonist comprises a VL having the sequence recited in SEQ ID NO:21 and a VH having the sequence recited in SEQ ID NO:26. In some embodiments, the PDL-1 antagonist comprises a VL having the sequence recited in SEQ ID NO:21 and a VH having the sequence recited in SEQ ID NO:27. In some embodiments, the PDL-1 antagonist comprises a VL having the sequence recited in SEQ ID NO:21 and a VH having the sequence recited in SEQ ID NO:28.

In some embodiments, the PDL-1 antagonist comprises a VL having the sequence recited in SEQ ID NO:22 and a VH having the sequence recited in any one of SEQ ID NOS:23-28. In some embodiments, the PDL-1 antagonist comprises a VL having the sequence recited in SEQ ID NO:22 and a VH having the sequence recited in SEQ ID NO:23. In some embodiments, the PDL-1 antagonist comprises a VL having the sequence recited in SEQ ID NO:22 and a VH having the sequence recited in SEQ ID NO:24. In some embodiments, the PDL-1 antagonist comprises a VL having the sequence recited in SEQ ID NO:22 and a VH having the sequence recited in SEQ ID NO:25. In some embodiments, the PDL-1 antagonist comprises a VL having the sequence recited in SEQ ID NO:22 and a VH having the sequence recited in SEQ ID NO:26. In some embodiments, the PDL-1 antagonist comprises a VL having the sequence recited in SEQ ID NO:22 and a VH having the sequence recited in SEQ ID NO:27. In some embodiments, the PDL-1 antagonist comprises a VL having the sequence recited in SEQ ID NO:22 and a VH having the sequence recited in SEQ ID NO:28.

In some embodiments, the PDL-1 antagonist comprises a VL having the sequence recited in any one of SEQ ID NOS:17-22 and a VH having the sequence recited in SEQ ID NO:23. In some embodiments, the PDL-1 antagonist comprises a VL having the sequence recited in any one of SEQ ID NOS:17-22 and a VH having the sequence recited in SEQ ID NO:24. In some embodiments, the PDL-1 antagonist comprises a VL having the sequence recited in any one of SEQ ID NOS:17-22 and a VH having the sequence recited in SEQ ID NO:25. In some embodiments, the PDL-1 antagonist comprises a VL having the sequence recited in any one of SEQ ID NOS:17-22 and a VH having the sequence recited in SEQ ID NO:26. In some embodiments, the PDL-1 antagonist comprises a VL having the sequence recited in any one of SEQ ID NOS:17-22 and a VH having the sequence recited in SEQ ID NO:27. In some embodiments, the PDL-1 antagonist comprises a VL having the sequence recited in any one of SEQ ID NOS:17-22 and a VH having the sequence recited in SEQ ID NO:28.

In additional embodiments, the PDL-1 antagonist competes with BMS-936559 (aka MDX-1105; Bristol-Myers Squibb) for binding to PDL-1. In additional embodiments, the PDL-1 antagonist binds to the same epitope of PDL-1 as BMS-936559. In particular embodiments, the PDL-1 antagonist used according to the disclosed methods is BMS-936559. See, e.g., Brahmer et al., N. Engl. J. Med. 366:2455-2465 (2012).

In additional embodiments, the PDL-1 antagonist competes with MPDL-3280A (aka RG₇₄₄6, Genentech/Roche) for binding to PDL-1. In additional embodiments, the PDL-1 antagonist binds to the same epitope of PDL-1 as MPDL-3280A. In particular embodiments, the PDL-1 antagonist used according to the disclosed methods is MPDL-3280A. See, e.g., Chen, D., Ann Oncol. 24 (suppl 1): i7 (2013).

### III. Methods of Treatment Using Antagonists of the PDL-1/PD-1 Interaction

As demonstrated and described herein, the antagonists of the PDL-1/PD-1 interaction (including, e.g., MEDI4736) are useful in therapeutic treatment methods, including the treatment of HPV-negative cancers. In certain embodiments, the antagonists are useful for inhibiting HPV-negative tumor growth, inducing differentiation of HPV-negative tumor cells, inhibiting metastases of HPV-negative tumors, reducing HPV-negative tumor volume, and/or reducing the tumorigenicity of an HPV-negative tumor, e.g., in in vivo methods.

Methods of determining whether a cancer is HPV-positive or HPV-negative are known.

In some embodiments, the HPV-negative cancer is squamous cell carcinoma of the head and neck (SCCHN).

In certain aspects, a patient presenting with a HPV-negative cancer is administered a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof. A PDL-1/PD-1 interaction antagonist, e.g. an anti-PDL-1 antibody or antigen binding fragment thereof (for example MEDI4736 or an antigen-binding fragment thereof) or an anti-PD-1 antibody or antigen binding fragment thereof can be administered only once or infrequently while still providing benefit to the patient. In further aspects the patient is administered additional follow-on doses. Follow-on doses can be administered at various time intervals depending on the patient's age, weight, clinical assessment, tumor burden, and/or other factors, including the judgment of the attending physician.

The intervals between doses can be every two weeks. The interval between doses can be every three weeks. The intervals between doses can be every two months (e.g., during a maintenance phase).

The dosing intervals can also be about every 14 days or about every 21 days. In some embodiments, “about” every 14 days or “about” every 21 days indicates 14 days +/−2 days or 21 days +/−2 days. In some embodiments, administration of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof is about every 14 to 21 days.

In some embodiments, at least two doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof is administered to the patient. In some embodiments, at least three doses, at least four doses, at least five doses, at least six doses, at least seven doses, at least eight doses, at least nine doses, at least ten doses, or at least fifteen doses or more can be administered to the patient. In some embodiments, a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof is administered over a two-week treatment period, over a four-week treatment period, over a six-week treatment period, over an eight-week treatment period, over a twelve-week treatment period, over a twenty-four-week treatment period, or over a one-year or more treatment period. In some embodiments, a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof is administered over a three-week treatment period, a six-week treatment period, over a nine-week treatment period, over a twelve-week treatment period, over a twenty-four-week treatment period, or over a one-year or more treatment period. In some embodiments, a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, is administered over a two-month treatment period, over a four-month treatment period, or over a six-month or more treatment period (e.g., during a maintenance phase).

The amount of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof to be administered to the patient will depend on various parameters such as the patient's age, weight, clinical assessment, tumor burden and/or other factors, including the judgment of the attending physician.

In certain aspects the patient is administered one or more doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 0.1 mg/kg. In certain aspects the patient is administered one or more doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 0.3 mg/kg. In certain aspects the patient is administered one or more doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 1 mg/kg. In certain aspects the patient is administered one or more doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 3 mg/kg. In certain aspects the patient is administered one or more doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 10 mg/kg. In certain aspects the patient is administered one or more doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 15 mg/kg.

In certain aspects the patient is administered at least two doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 0.1 mg/kg. In certain aspects the patient is administered at least two doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 0.3 mg/kg. In certain aspects the patient is administered at least two doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 1 mg/kg. In certain aspects the patient is administered at least two doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 3 mg/kg. In certain aspects the patient is administered at least two doses of a PDL-1/PD-1 interaction antagonist, e.g. an anti-PDL-1 antibody or antigen binding fragment thereof (e.g. MED14736 or an antigen-binding fragment thereof) or an anti-PD-1 antibody or antigen binding fragment thereof, wherein the dose is about 10 mg/kg. In certain aspects the patient is administered at least two doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 15 mg/kg. In some embodiments, the at least two doses are administered about two weeks apart. In some embodiments, the at least two doses are administered about three weeks apart.

In certain aspects the patient is administered at least three doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 0.1 mg/kg. In certain aspects the patient is administered at least three doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 0.3 mg/kg. In certain aspects the patient is administered at least three doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 1 mg/kg. In certain aspects the patient is administered at least three doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 3 mg/kg. In certain aspects the patient is administered at least three doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 10 mg/kg. In certain aspects the patient is administered at least three doses of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, wherein the dose is about 15 mg/kg. In some embodiments, the at least three doses are administered about two weeks apart. In some embodiment, the at least three doses are administered about three weeks apart.

In certain aspects, administration of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, according to the methods provided herein is through parenteral administration. For example, a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof can be administered by intravenous infusion or by subcutaneous injection. In some embodiments, the administration is by intravenous infusion.

In certain aspects, a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof is administered according to the methods provided herein in combination or in conjunction with additional cancer therapies. Such therapies include, without limitation, chemotherapeutic agents such as Vemurafenib, Erlotinib, Afatinib, Cetuximab, Carboplatin, Bevacizumab, Erlotinib, or Pemetrexed, or other chemotherapeutic agents, as well radiation or any other anti-cancer treatments.

The methods provided herein can decrease tumor size, retard tumor growth or maintain a steady state. In certain aspects the reduction in tumor size can be significant based on appropriate statistical analyses. A reduction in tumor size can be measured by comparison to the size of patient's tumor at baseline, against an expected tumor size, against an expected tumor size based on a large patient population, or against the tumor size of a control population. In certain aspects provided herein, the administration of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof can reduce a tumor size by at least 25%. In certain aspects provided herein, the administration of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof can reduce a tumor size by at least 25% within about 6 weeks of the first treatment. In certain aspects provided herein, the administration of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof can reduce a tumor size by at least 25% within about 12 weeks of the first treatment. In certain aspects provided herein, the administration of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof can reduce a tumor size by at least 25% within about 18 weeks of the first treatment.

In certain aspects, use of the methods provided herein, i.e., administration of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, can decrease tumor size within 6 weeks, within 7 weeks, within 8 weeks, within 9 weeks, within 10 weeks, within 12 weeks, within 16 weeks, within 20 weeks, within 24 weeks, within 28 weeks, within 32 weeks, within 36 weeks, within 40 weeks, within 44 weeks, within 48 weeks, or within 52 weeks of the first treatment.

The methods provided herein can decrease or retard tumor growth. In some aspects the reduction or retardation can be statistically significant. A reduction in tumor growth can be measured by comparison to the growth of patient's tumor at baseline, against an expected tumor growth, against an expected tumor growth based on a large patient population, or against the tumor growth of a control population.

In certain aspects, a patient achieves disease control (DC). Disease control can be a complete response (CR), partial response (PR), or stable disease (SD).

A “complete response” (CR) refers to the disappearance of all lesions, whether measurable or not, and no new lesions. Confirmation can be obtained using a repeat, consecutive assessment no less than four weeks from the date of first documentation. New, non-measurable lesions preclude CR.

A “partial response” (PR) refers to a decrease in tumor burden≧50% relative to baseline. Confirmation can be obtained using a consecutive repeat assessment at least 4 weeks from the date of first documentation

“Progressive disease” (PD) refers to an increase in tumor burden≧25% relative to the minimum recorded (nadir). Confirmation can be obtained by a consecutive repeat assessment at least 4 weeks from the date of first documentation. New, non-measurable lesions do not define PD.

“Stable disease” (SD) refers to not meeting the criteria for CR, PR, or PD.

In certain aspects, administration of a PDL-1/PD-1 interaction antagonist, e.g. an anti-PDL-1 antibody or antigen-binding fragment thereof (e.g. MEDI4736 or an antigen-binding fragment thereof) or an anti-PD-1 antibody or antigen-binding fragment thereof can increase progression-free survival (PFS).

In certain aspects, administration of a PDL-1/PD-1 interaction antagonist, e.g. an anti-PDL-1 antibody or antigen binding fragment thereof (e.g. MEDI4736 or an antigen-binding fragment thereof) or an anti-PD-1 antibody or antigen binding fragment thereof, can increase overall survival (OS).

In some embodiments, the patient has previously received treatment with at least one chemotherapeutic agent. In some embodiments, the patient has previously received treatment with at least two chemotherapeutic agents. The chemotherapeutic agent can be, for example, and without limitation, Vemurafenib, Erlotinib, Afatinib, Cetuximab, Carboplatin, Bevacizumab, Erlotinib, and/or Pemetrexed.

In some embodiments, the tumor is refractory or resistant to at least one chemotherapeutic agent. In some embodiments, the tumor is refractory or resistant to at least two chemotherapeutic agents. The tumor can be refractory or resistant to one or more of, for example, and without limitation, Vemurafenib, Erlotinib, Afatinib, Cetuximab, Carboplatin, Bevacizumab, Erlotinib, and/or Pemetrexed.

In some embodiments, the patient has an Eastern Cooperative Oncology Group (ECOG) (Oken M M, et al. Am. J. Clin. Oncol. 5: 649-55 (1982)) performance status of 0 or 1 prior to the administration of MEDI4736 or an antigen-binding fragment thereof.

In some embodiments, the patient has an Eastern Cooperative Oncology Group (ECOG) (Oken M M, et al. Am. J. Clin. Oncol. 5: 649-55 (1982)) performance status of 0 or 1 prior to the administration of MEDI4736 or an antigen-binding fragment thereof.

As discussed herein, in some embodiments, the antagonist of the PLD-1/PD-1 interaction is MEDI4736 or an antigen-binding fragment thereof. In some embodiments, administration of MEDI4736 or an antigen-binding fragment thereof can result in desirable pharmacokinetic parameters. Total drug exposure can be estimated using the “area under the curve” (AUC). “AUC (tau)” refers to AUC until the end of the dosing period, whereas “AUC (inf)” refers to the AUC until infinite time. The administration can produce AUC (tau) of about 100 to about 2,500 d·μg/mL. The administration can produce a maximum observed concentration (Cmax) of about 15 to about 350 μg/mL. The half-life of MEDI4736 or an antigen-binding fragment thereof can be about 5 to about 25 days. In addition, the clearance of MEDI4736 or an antigen-binding fragment thereof can be about 1-10 ml/day/kg.

As discussed herein, in some embodiments, the antagonist of the PLD-1/PD-1 interaction is an anti-PDL-1 antibody or antigen binding fragment thereof (e.g. MEDI4736 or an antigen-binding fragment thereof). In some embodiments, administration of an anti-PDL-1 antibody or antigen-binding fragment thereof (e.g. MEDI4736 or an antigen-binding fragment thereof) can decrease free PDL-1 levels. Free PDL-1 refers to PDL-1 that is not bound (e.g., by MEDI4736). In some embodiments, PDL-1 levels are reduced by at least 80%. In some embodiments, PDL-1 levels are reduced by at least 90%. In some embodiments, PDL-1 levels are reduced by at least 95%. In some embodiments, PDL-1 levels are reduced by at least 99%. In some embodiments, PDL-1 levels are eliminated following administration of an anti-PDL-1 antibody or antigen binding fragment thereof (e.g. MEDI4736 or an antigen-binding fragment thereof). In some embodiments, administration of an anti-PDL-1 antibody or antigen binding fragment thereof (e.g. MEDI4736 or an antigen-binding fragment thereof) reduces the rate of increase of PDL-1 levels as compared, e.g., to the rate of increase of PDL-1 levels prior to the administration of an anti-PDL-1 antibody or antigen binding fragment thereof (e.g. MEDI4736 or an antigen-binding fragment thereof).

### EXAMPLES

### Example 1

### Patients and Methods

### (a) Subjects

Subjects in this study were required to be 18 years of age or older with advanced malignant melanoma, renal cell carcinoma (RCC), non-small cell lung cancer (NSCLC), or colorectal cancer (CRC) refractory to standard therapy or for which no standard therapy exists. Subjects in the dose-expansion phase of the study will be adults with advanced malignant melanoma, NSCLC, or CRC refractory to standard therapy or for which no standard therapy exists. Additional subjects in the dose-expansion phase had NSCLC (Squamous cell carcinoma), hepatocellular cancer (HCC), triple-negative breast cancer (TNBC), pancreatic cancer, GI cancer, melanoma, uveal melanoma, or Squamous cell carcinoma of the head and neck (SCCHN). The cancers must be histologically- or cytologically confirmed. The subjects are required to have an Eastern Cooperative Oncology Group (ECOG) status of 0 or 1 as well as adequate organ and marrow function. Adequate organ and marrow function was defined as: hemoglobin≧9 g/dL; absolute neutrophil count≧1,500/mm³; lymphocyte count≧800/mm³; platelet count≧100,000/mm³; aspartate aminotransferase (AST) and alanine aminotransferase (ALT)≧2.5×institutional upper limit of normal (ULN); bilirubin≧1.5×ULN except in the case of subjects with documented or suspected Gilbert's disease (for these subjects, bilirubin must be ≧5×ULN); creatinine clearance≦50 mL/min as determined by the Cockcroft-Gault equation or by 24-hour urine collection for determination of creatinine clearance.

Subjects are not able to participate if they have active autoimmune disease, prior anti-PD-1 or anti-PDL-1 therapy, or prior severe or persistent immune-related adverse events (irAE). Subjects are not permitted to have any concurrent chemotherapy, immunotherapy, biologic or hormonal therapy for cancer treatment, but concurrent use of hormones for non-cancer related conditions (e.g., insulin for diabetes and hormone replacement therapy) are allowed.

### (b) Design of the Study

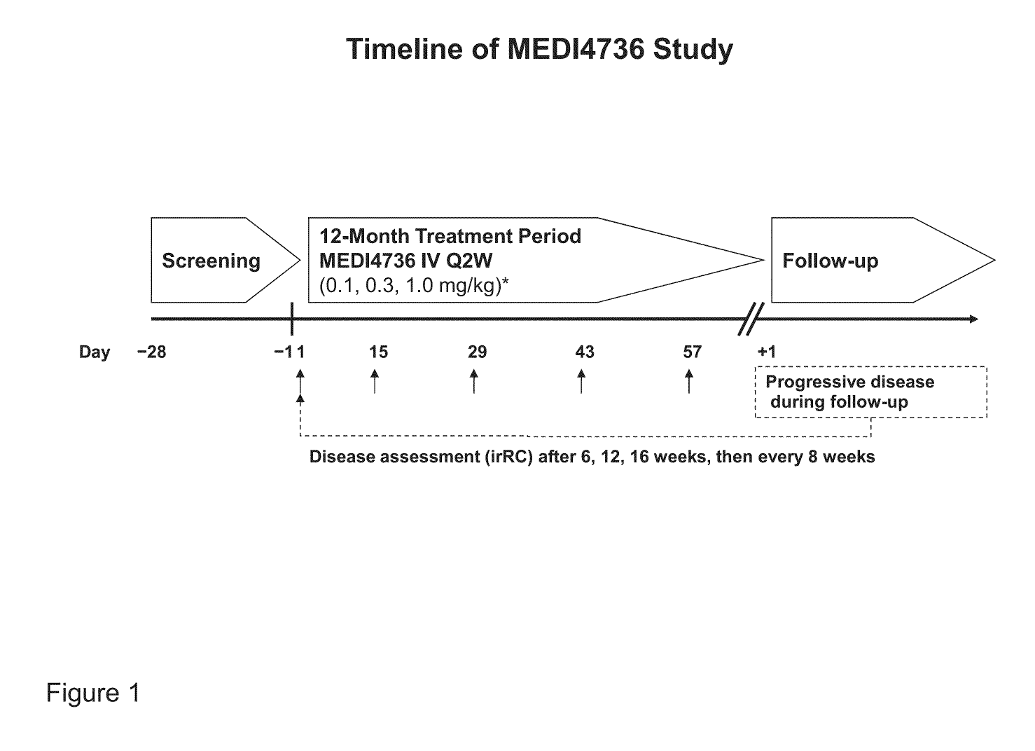

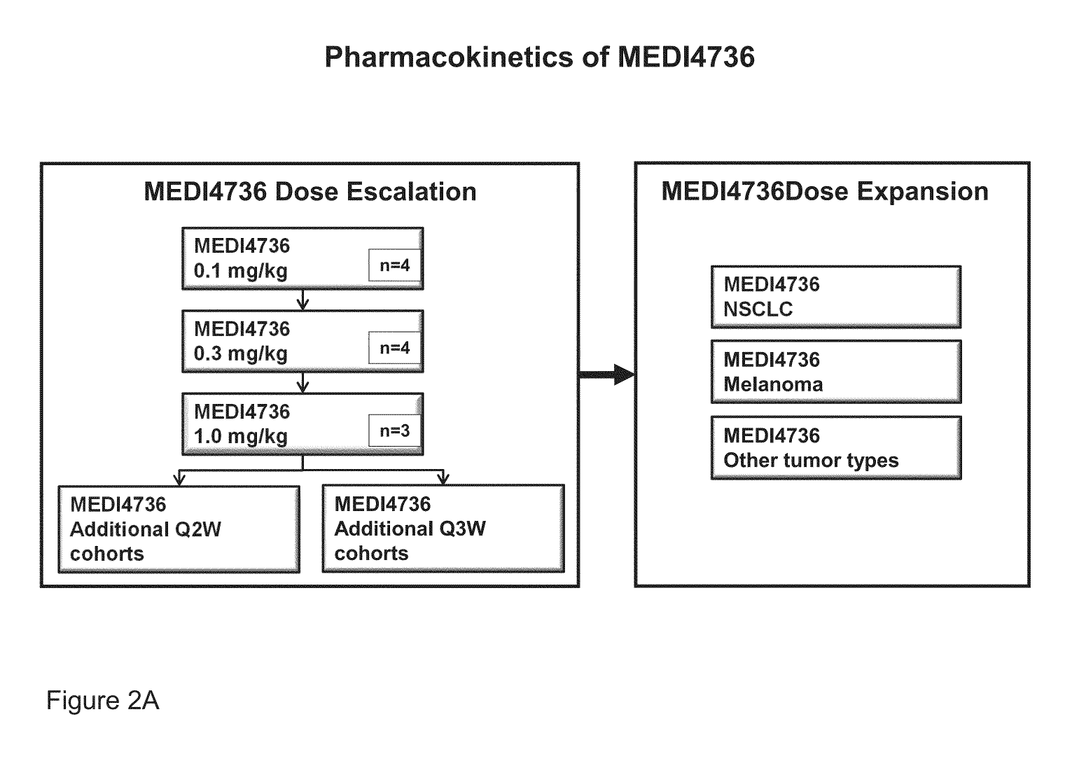

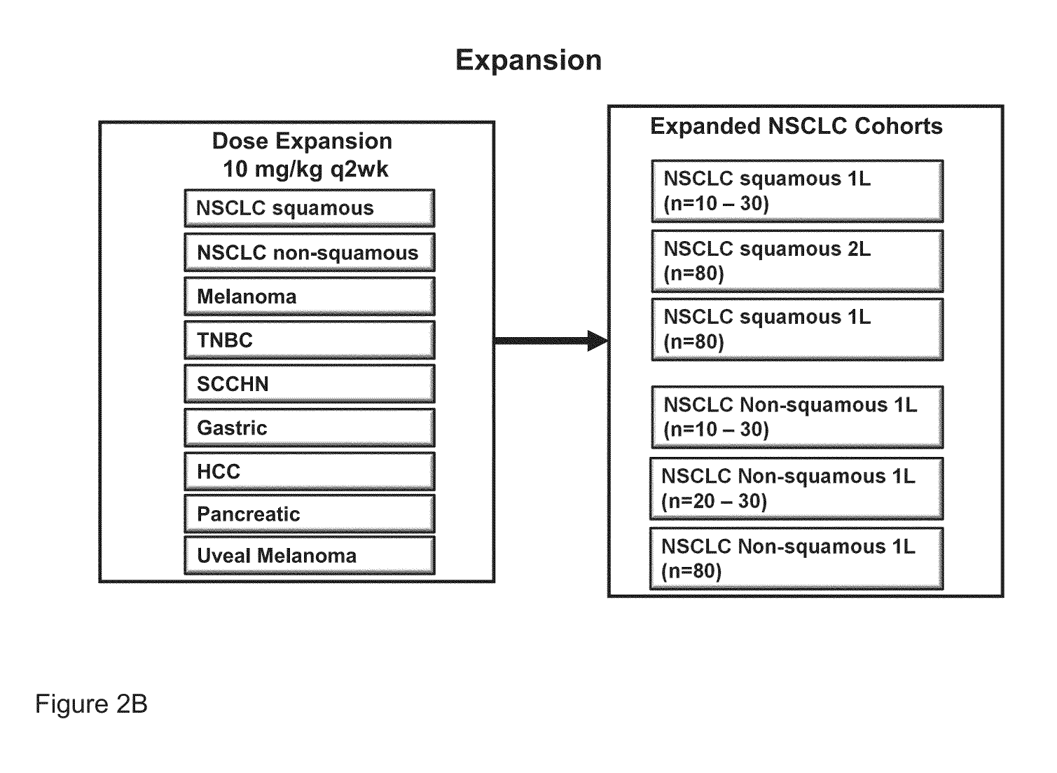

The study is a multicenter, open-label, Phase 1, first-time-in-human, dose-escalation and dose-expansion study in which multiple doses of MEDI4736 are administered via intravenous (IV) infusion to cancer patients. MEDI4736 was administered at 0.1, 0.3, 1, 3, 10, and 15 mg/kg doses. The study flow diagram is shown in FIG. 1. The first day of dosing is considered Day 1, and diseases assessment takes place after 6, 12, and 16 weeks, and then every 8 weeks.

A dose-escalation was performed with administration every 2 weeks (Q2W) (+/−2 days) to different cohorts with doses of 0.1, 0.3, 1, 3, and 10 mg/kg doses.

A separate dose-escalation was performed with administration every 3 weeks (Q3W) at 15 mg/kg. An expansion phase is then conducted using the maximum tolerated dose (MTD) or optimal biological dose (OBD) identified in the dose-escalation.

### Dose Escalation

In the dose-escalation phase, the first dose of MEDI4736 was administered to all subjects in the first cohort as a 0.1 mg/kg infusion given over 4 hours. Subsequent infusions (2nd and 3rd doses, etc.) for the first cohort were given over 60 minutes Q2W. The doses for subsequent cohorts were 0.3, 1.0, 3.0, or 10 mg/kg, administered as a 60-minute IV infusion Q2W. A summary of the dose cohorts for the initial dose escalation is provided in Table 1 below. Additional doses of 15 mg/kg were also administered at Q3W.

TABLE 1
Q2W Dosing Regimen

| Dose | Number | |
|---|---|---|
| Cohort | Subjects | Dosing Regimen |
| 1 | 3-6 | 0.1 mg/kg as a 4-hour IV infusion for the initial |
| | | dose, and then as 60-minute IV infusion once every |
| | | 2 weeks |
| 2 | 3-6 | 0.3 mg/kg as a 60-minute IV infusion once every |
| | | 2 weeks |
| 3 | 3-6 | 1.0 mg/kg as a 60-minute IV infusion once every |
| | | 2 weeks |
| 4 | 3-6 | 3.0 mg/kg as a 60-minute IV infusion once every |
| | | 2 weeks |
| 5 | 3-6 | 10 mg/kg as a 60-minute IV infusion once every |
| | | 2 weeks |
| 6 | 3-6 | 15 mg/kg as a 60-minute IV infusion once every |
| | | 3 weeks |

With the completion of all cohorts in the Q2W dose escalation regimen, a separate dose escalation using the Q3W regimen begins and proceeds to a dose of up to 15 mg/kg Q3W based on available safety, PK/pharmacodynamics, and clinical data. The starting dose in the Q3W escalation is the equivalent dosing rate (in average mg/kg/week) to the optimal biological dose (OBD) (or highest dose tested if an OBD is not identified).

Subjects in the dose-escalation phase continue treatment until confirmed PD, initiation of alternative cancer therapy, unacceptable toxicity, or other reasons to discontinue treatment occur. In those subjects achieving confirmed disease control (DC), treatment may continue until 6 months past the date of confirmed DC. DC will include stable disease (SD) with a duration of 3 or more months, partial response (PR), and complete response (CR).

### Dose Expansion

Following the completion of dose escalation at Q2W and Q3W, the dose regimen for the expansion phase is selected. Subjects enrolled in the dose expansion cohorts will receive MEDI4736 at the maximum tolerated dose (MTD), optimal biological dose (OBD), or the highest dose evaluated during dose escalation if no MTD or OBD is determined, given as an IV infusion at the selected dose and frequency. Subjects who achieve disease control (DC) will continue treatment and then enter the maintenance period. Upon evidence of progressive disease (PD) at any time during the maintenance period, MEDI4736 will be re-administered as an IV infusion until confirmed PD or other reason to discontinue MEDI4736.

### Maintenance Period

Subjects who achieve disease control (DC) during the escalation or expansion phases enter the maintenance period in which treatment can continue until six months past the date of confirmed DC.

During the maintenance period, MEDI4736 is administered as an IV infusion every 2 months for 6 months. Physical examination of subjects will be performed at months 2, 4, and 6. After a 6-month period of every 2-month dosing, MEDI4736 is discontinued. Upon evidence of progressive disease (PD), MEDI4736 is re-administered as an IV infusion at a Q2W or Q3W schedule until confirmed PD, initiation of alternative cancer therapy, unacceptable toxicity, withdrawal of consent, or other reason to discontinue treatment, for a maximum of 2 years.

### (c) Phamacokinetic, Anti-Tumor and Safety Assessments

Measurement of MEDI4736 concentrations in serum was performed using a validated immunoassay during the Q2W dose-escalation phase. Blood samples for pharmacokinetic assessment, as well as for soluble PDL-1 (sPDL-1) concentrations, were collected according to the following schedules during the Q2W dose-escalation phase:
First dose: Day 1 predose, end of infusion (EOI), and 3 hours after EOI, and Days 2, 3, 5, and 10 (+/−1 day). An additional sample at 2 hours after the start of the infusion was taken during the first study subject's initial, 4-hour infusion.
Second dose: Predose, EOI, 3 hours after EOI, and Day 8.
Subsequent even-numbered doses only: Predose and EOI.
Upon discontinuation or last dose, a pharmacokinetic (PK) sample should be drawn at 14 days, 30 days, 2 and 3 months after last dose.

For Q3W dosing, the pharmacokinetic assessments are performed at the same schedule as Q2W dosing except that a blood sample is also collected on Day 15 after the first dose. During the dose-expansion phase, pharmacokinetic assessments are performed every two months (Day 1 predose and EOI). In addition, upon discontinuation or last dose, a pharmacokinetic (PK) sample is drawn at 14 days, 30 days, 2 months, and 3 months after the last dose. During the maintenance phase, pharmacokinetic assessments and evaluations of sPDL-1 are performed on Days 14 and 30 (+/−3 days), and at months 2, 4, and 6 (+/−1 week).

The presence of anti-drug antibodies (ADA) was assessed (and will continue to be assessed) on Day 1 (preinfusion) and at all doses following dose 2 during the Q2W dose-escalation phase. ADA will be assessed according to the same schedule in the Q3W dose-escalation and dose-expansion phases. During the maintenance phase, ADA will be assessed at month 6 (+/−1 week).

Tumor assessments were performed (and will continue to be performed) during screening (day −28 to day −1) and at week 7 in the Q2W dose-escalation phase. Tumor assessments are performed with the same timing in the Q3W dose-escalation phase and the dose-expansion phase. Tumor assessments can include the following evaluations: physical examination (with photograph and measurement of skin lesions as applicable), CT, or MRI scan of the chest, abdomen, and pelvis, and CT or MRI scan of the brain. Computed tomography or MRI scan of the brain is performed only at screening or if the subject is neurologically symptomatic. During the maintenance phase, tumor assessments are performed at months 2, 4, and 6 (+/−1 week).

During the expansion phase, tumor biopsies are also performed during screening (day −28 to day −1) and at week 7.

Assessments of anti-tumor activity are based on the immune-related objective response rate (ORR), immune-related disease control rate (DCR), immune-related duration of response (DR), immune-related progression-free survival (PFS), and overall survival (OS). Immune-related response criteria (Wolchok et al., Clin Cancer Res. 15:7412-20 (2009)) were used to determine tumor response.

The ORR is defined as the proportion of subjects with confirmed complete response (CR) or confirmed partial response (PR). Confirmed responses are those that persist on repeat imaging study≧4 weeks after the initial documentation of response. The DCR is defined as the proportion of subjects with CR, PR or stable disease (SD) (subjects achieving SD will be included in the DCR if they maintain SD for ≧3 months). The 95% confidence interval (CI) of ORR and DCR is estimated using the exact probability method. The duration of response (DR) is the duration from the first documentation of objective response to the first documented disease progression. Progression-free survival (PFS) is measured from the start of treatment with MEDI4736 until the documentation of confirmed immune-related disease progression or death due to any cause, whichever occurs first. Overall survival (OS) is the time from the start of treatment with MEDI4736 until death.

Adverse events are monitored following administration of MEDI4736. Other assessments include physical examination, vital sign monitoring, and laboratory measurements.

### Example 2

### Results

### (a) Enrollment and Baseline Characteristics

The baseline characteristics of the subjects administered 0.1, 0.3, or 1 mg/kg MEDI4736 in the Q2W dose-escalation phase are provided in Table 2 below.

TABLE 2
Demographics for Q2W dosing

| | Characteristic | | | |
|---|---|---|---|---|
| | 0.1 mg/kg | 0.3 mg/kg | 1.0 mg/kg | Total |
| | (n = 4) | (n = 4) | (n = 3) | (N = 11) |
| Mean Age (yrs) | 58.5 (46-65) | 68.0 (65-71) | 65.3 (43-77) | 63.8 (43-77) |
| Gender | 2/2 | 3/1 | 1/2 | 6/5 |
| (male/female) | | | | |
| ECOG 1 at | 2 | 1 | 2 | 5 |
| baseline (n) | | | | |
| ECOG 0 at | 2 | 3 | 1 | 6 |
| baseline (n) | | | | |
| Mean number | 9.8 (5-17) | 5.8 (4-9) | 6.0 (1-10) | 7.3 (1-17) |
| of prior cancer | | | | |
| treatments | | | | |
| (range) | | | | |
| Colorectal | 0 | 1 | 0 | 1 |
| tumor (n) | | | | |
| Melanoma (n) | 1 | 0 | 1 | 2 |
| NSCLC (n) | 3 | 3 | 2 | 8 |

### (b) Pharmacokinetics

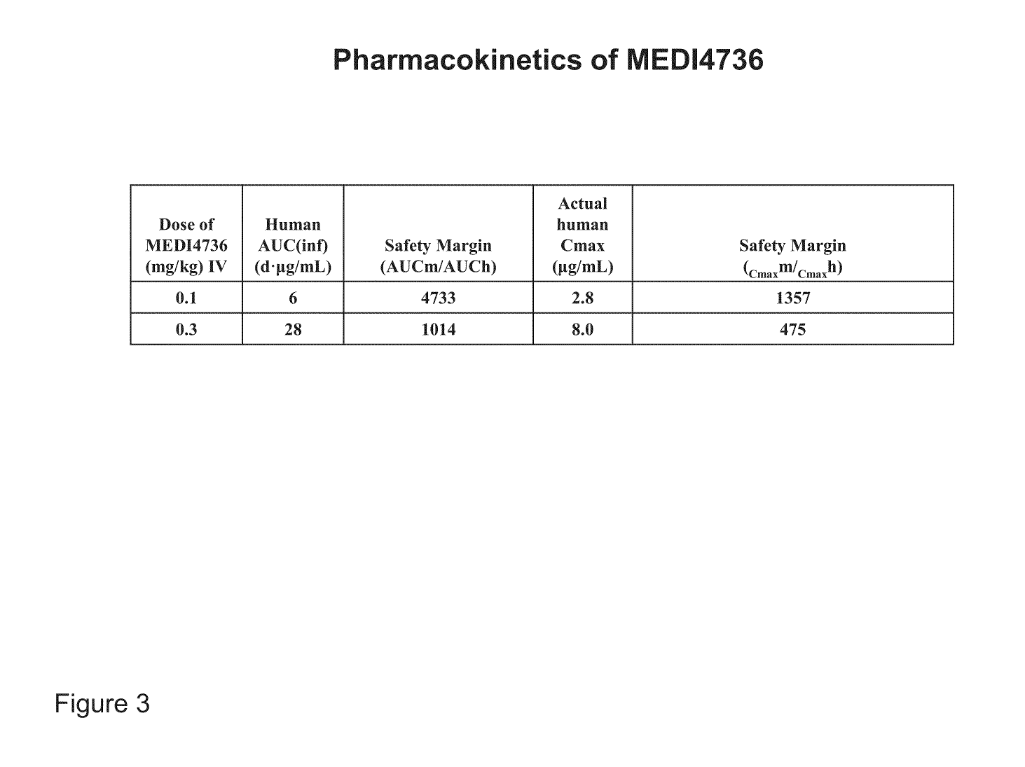

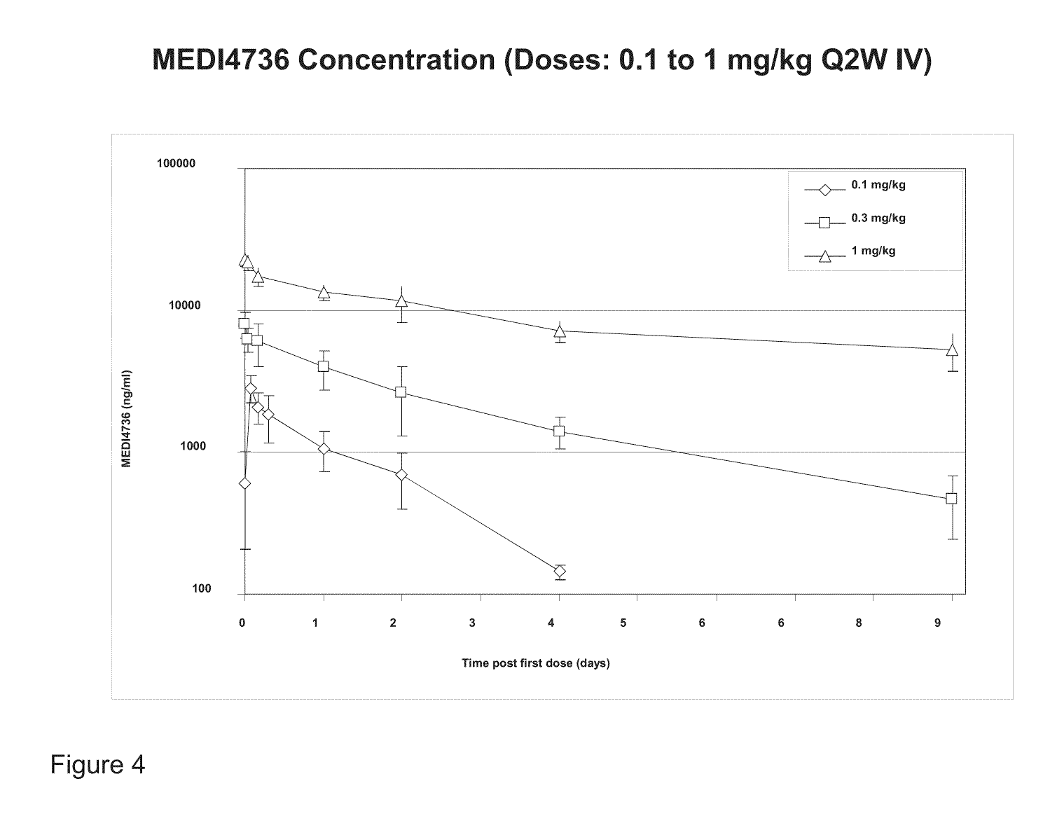

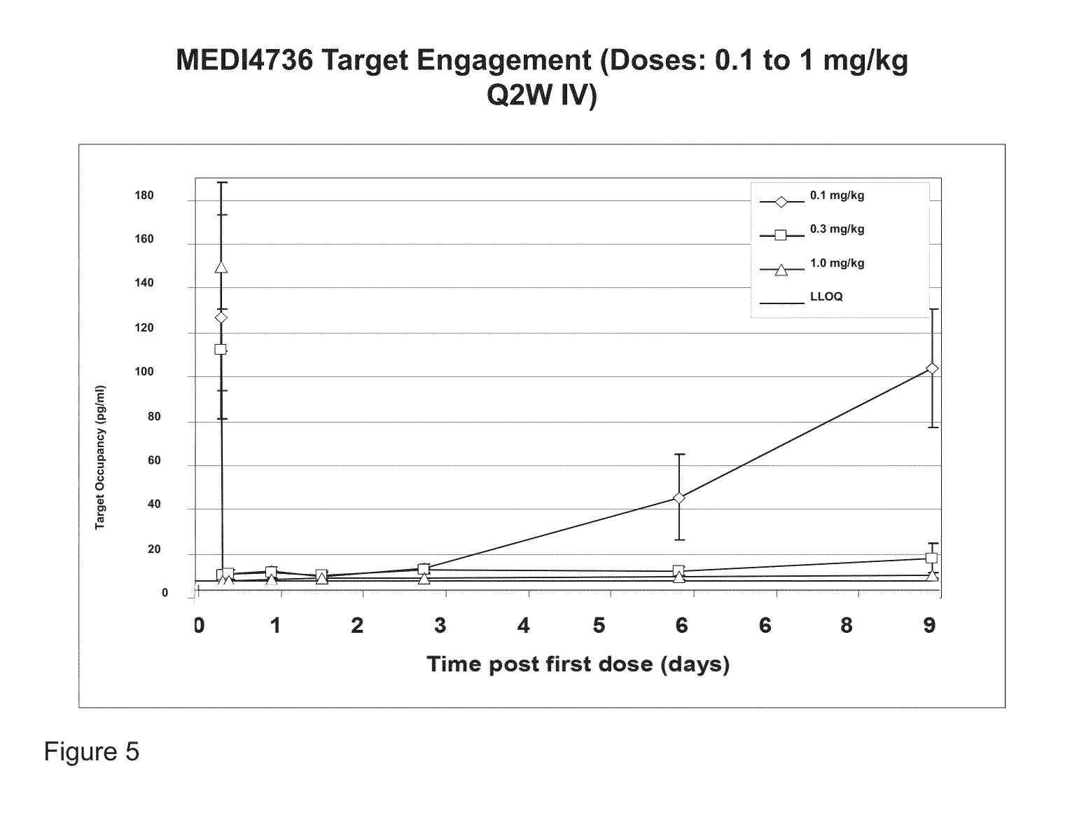

The pharmacokinetic data resulting from administration of MEDI4736 at 0.1 and 0.3 mg/kg in the Q2W dose-escalation phase is summarized in FIG. 3. MEDI4736 exhibited a non-linear PK at lower doses, but a linear PK with doses ≧1.0 mg/kg Q2W. See FIG. 4. MEDI4736 also showed a dose-dependent increase in target engagement, consistent with binding of MEDI4736 with PDL-1. Based on calculations using pK data and measurements of soluble PDL-1, significant target occupancy was achieved with doses ≧0.3 mg/kg Q2W, and near complete saturation is expected at doses ≧3 mg/kg Q2W. See FIG. 5.

### (c) Efficacy

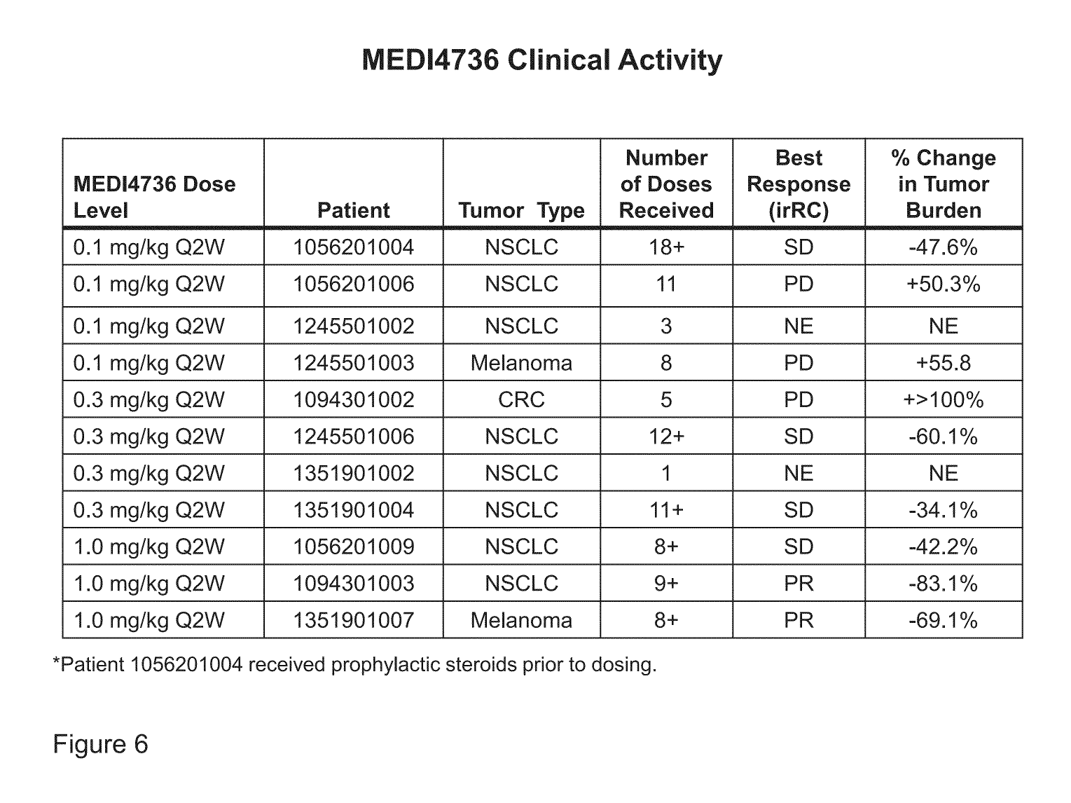

Tumor shrinkage was observed at all dose levels, including in heavily pretreated patients and in patients with large tumor burdens. Activity was apparent quickly (6 weeks) and was durable. Partial responses (PR) and stable disease (SD) were observed in patients receiving as little as 0.1 mg/kg Q2W. See FIG. 6 and Table 3 below.

| | | | Number | | % Change |
|---|---|---|---|---|---|
| Dose | Dosing | | of Doses | Best | in Tumor |
| (mg/kg) | Frequency | Subject ID | Received | Response | Burden |
| 0.1 | Q2W | 1056201004 | 25 | SD | −47.6 |
| 0.1 | Q2W | 1056201006 | 11 | PD | 50.3 |
| 0.1 | Q2W | 1245501002 | 3 | NE | NE |
| 0.1 | Q2W | 1245501003 | 8 | PD | 55.8 |
| 0.3 | Q2W | 1094301002 | 5 | PD | +>100 |
| 0.3 | Q2W | 1245501006 | 24 | PR | −60.1 |
| 0.3 | Q2W | 1351901002 | 1 | NE | NE |
| 0.3 | Q2W | 1351901004 | 22 | PR | −71.2 |
| 1 | Q2W | 1056201009 | 19 | SD | −46.6 |
| 1 | Q2W | 1094301003 | 18 | PR | −83.3 |
| 1 | Q2W | 1351901007 | 17 | PR | −76.8 |
| 3 | Q2W | 1056201010 | 5 | SD | −16.1 |
| 3 | Q2W | 1094301004 | 7 | PD | 38 |
| 3 | Q2W | 1351901008 | 3 | PD | +>100 |
| 10 | Q2W | 1002501208 | 5 | SD | 32.4 |
| 10 | Q2W | 1056201201 | 5 | PD | +>100 |
| 10 | Q2W | 1094301205 | 13 | SD | 9.3 |
| 10 | Q2W | 1245501206 | 5 | PD | 60 |
| 10 | Q2W | 1351901209 | 3 | PD | 82 |
| 10 | Q2W | 1371501207 | 2 | PD | 75.1 |
| 15 | Q3W | 1002501313 | 1 | NA | NA |
| 15 | Q3W | 1056201213 | 4 | SD | 16.4 |
| 15 | Q3W | 1245501211 | 5 | SD | −5 |
| 15 | Q3W | 1351901223 | 4 | SD | 10 |
| 15 | Q3W | 1371501297 | 2 | NA | NA |
| 15 | Q3W | 1372001228 | 5 | SD | 0 |

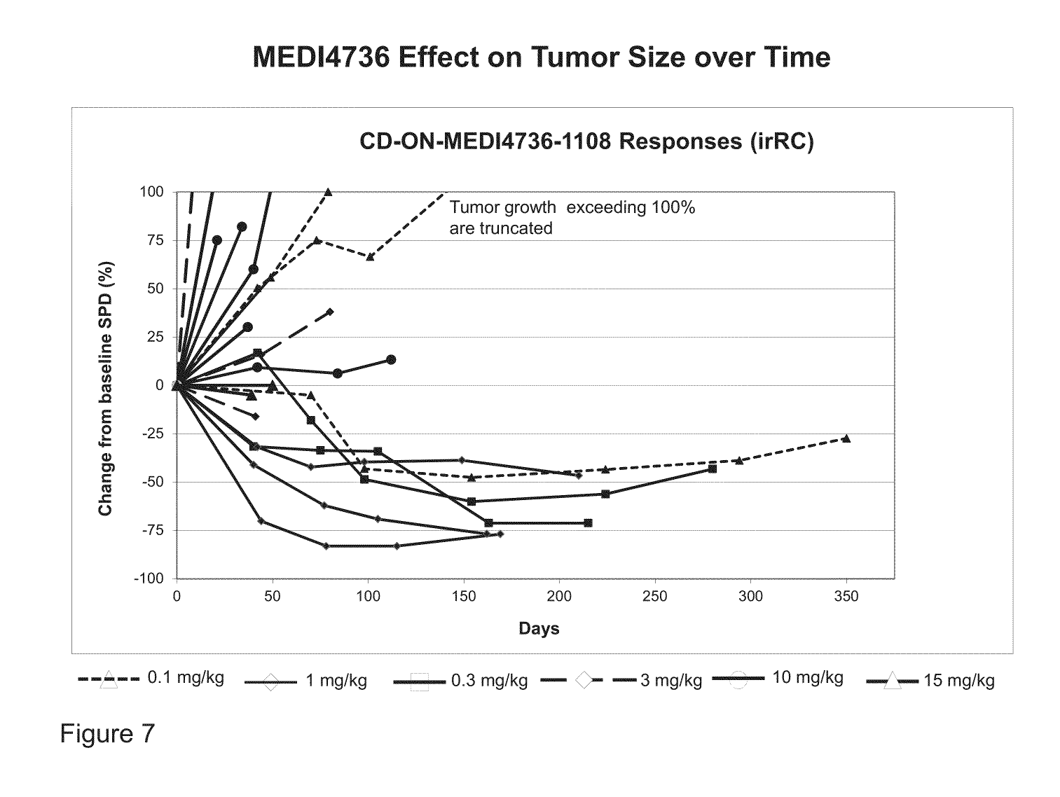

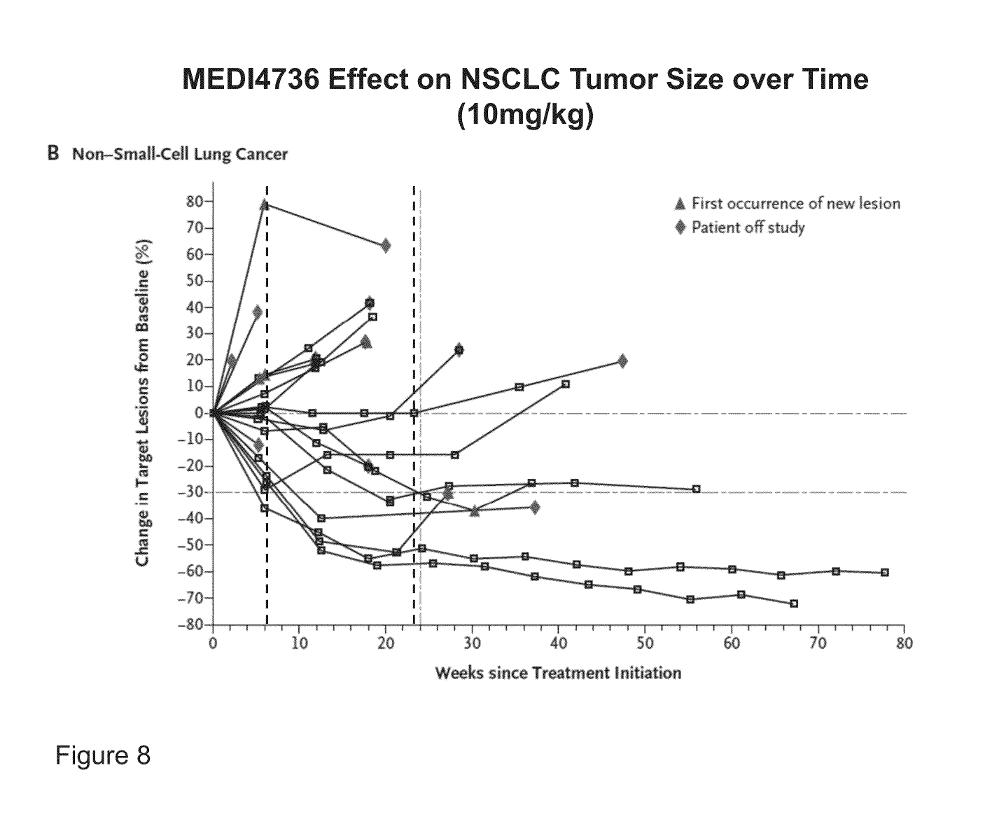

In addition, tumor burdens decreased as must as 83% in patients receiving up to 10 mg/kg Q2W. See FIGS. 6-8. For instance, one NSCLC adenocarcinoma patient (1351901004) receiving 0.3 mg/kg showed a 31% decrease in tumor burden after 6 weeks and a 71% decrease in tumor burden after 23 weeks. Prophylactic steroids were used in one subject and did not appear to affect clinical activity.

In the dose-expansion phase, clinical activity was initially observed in subjects with non-small cell lung cancer, melanoma, and pancreatic cancer. Stable disease (at 12 weeks) was observed in subjects with non-small cell lung cancer (non-squamous), pancreatic cancer, GI cancer, melanoma, and squamous cell carcinoma of the head and neck.

### (d) Safety and Anti-Drug Antibodies

MEDI4736 was generally well tolerated. No pneumonitis, colitis (of any grade), or hyperglycemia was observed. In addition, no treatment-related Grade≧3 events were observed and no dose-limiting toxicities were observed.

An extremely low incidence of ADAs was observed over the dose range of 0.1 to 3 mg/kg. In particular, only 1 of 15 patients who received a dose of dose range of 0.1 to 1 mg/kg tested ADA positive with PK/PD implications.

### (e) Discussion

This study demonstrates that MEDI4736 has favorable pK properties and is generally well tolerated. In addition, MEDI4736 is effective in treating tumors (including melanoma and non-small cell lung cancer) while producing a low incidence of ADA.

### Example 3

### Correlation of HPV Status and Treatment Efficacy

The efficacy of several antibody therapeutics has been shown to be correlated with antigen expression level. For example, Herceptin® (trastuzumab) binds to HER2 protein, and data from efficacy trials with Herceptin®shows that beneficial treatment effects were largely limited to patients with the highest levels of HER2 protein expression. The degree of HER2 overexpression is considered a predictor of treatment effect, and Herceptin® is specifically indicated for cancers overexpressing HER2.

Increased levels of PD-1 and PDL-1 have been observed in HPV-positive tumors. Therefore, the efficacy of MEDI4736 in treating HPV-positive and HPV-negative tumors was examined to determine if HPV-positive tumor status was a predictor of treatment effect. In these experiments, the HPV status of twelve squamous cell carcinoma of the head and neck (SCCHN) tumors was determined. Four of the twelve patients had HPV-positive tumors and eight of the twelve patients had HPV-negative tumors. PDL-1 status was also assessed. Two of the twelve subjects were PDL-1-positive, and eight of the subjects were PDL-1 negative (the PDL-1 status of two of the subjects was not available).

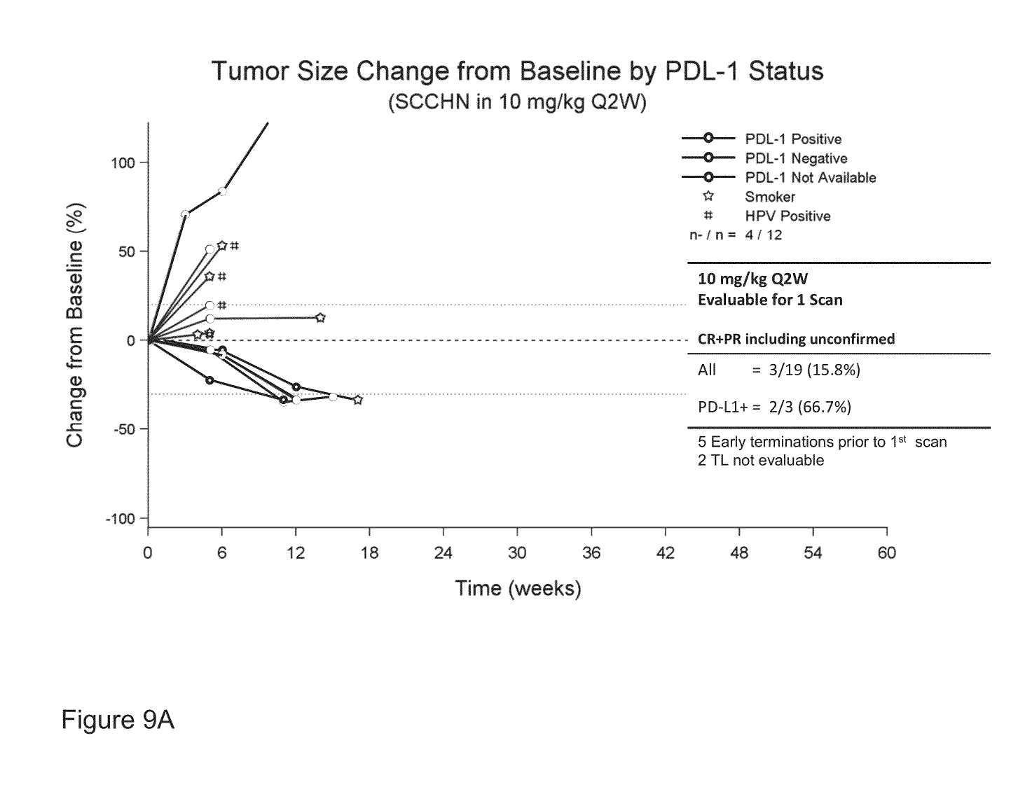

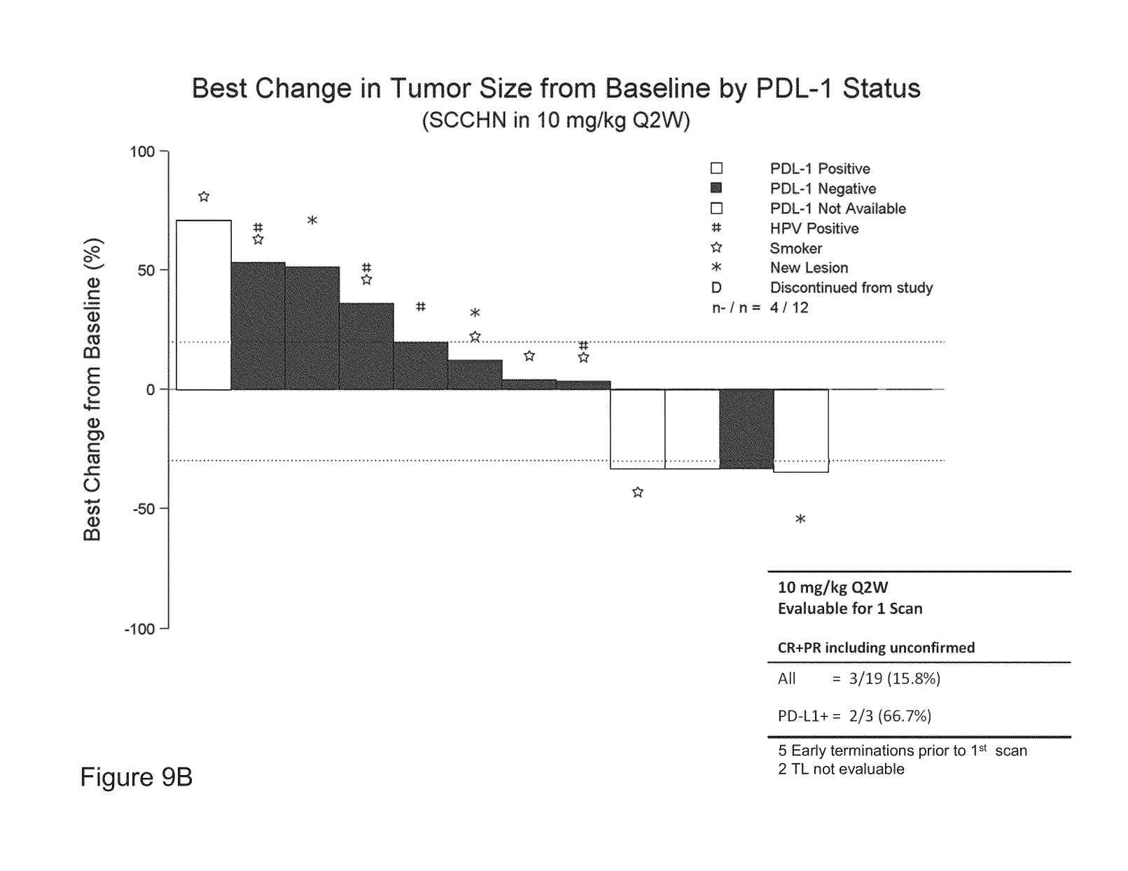

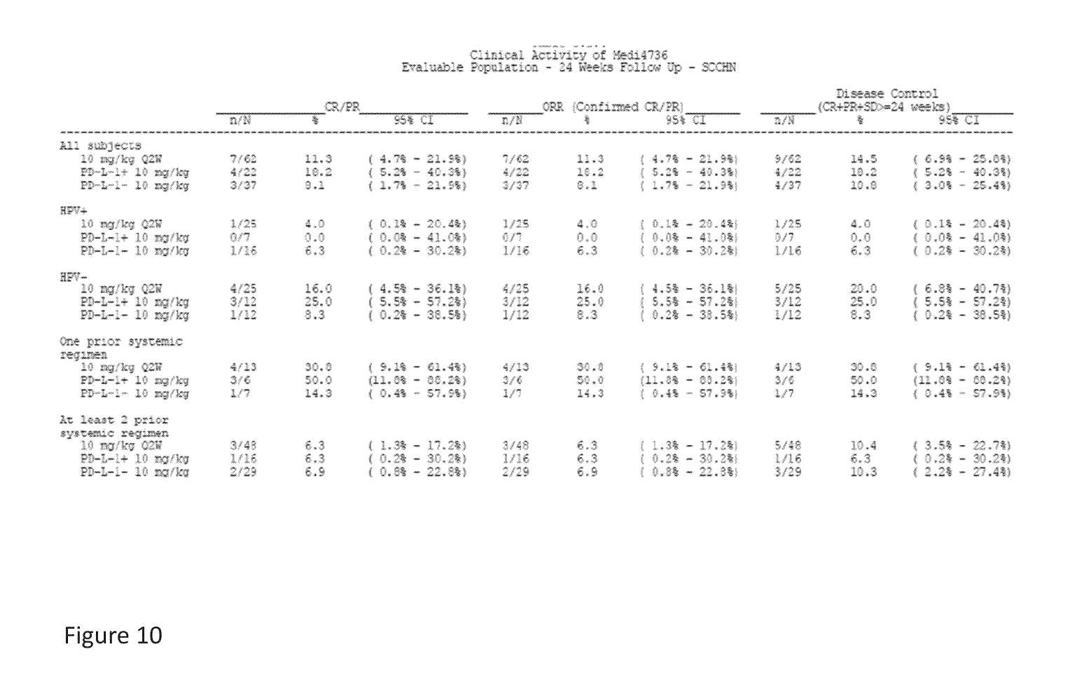

Tumor size was measured before treatment with MEDI4736 and at weeks 6, 12, and 18 after treatment. The results are shown in FIGS. 9A and 9B. Tumor shrinkage was observed in 4 of the 12 subjects following administration of 10 mg/kg Q2W of MED14736. In all 4 of these patients, tumors shrank by at least 25%. Two of those patients were PDL-1 positive, one was PDL-1 negative, and the PDL-1 status of the fourth was unknown. In addition, a complete responses (CR) and partial responses (PR) was observed in 3/19 (15.8%) of subjects in an even higher percentage in the patients who were PDL-1 positive (⅔; 66.7%). FIG. 10 summarizes the response of subjects treated with MEDI4736 (including 24 months of follow-up). Thus, MEDI4736 was effective in treating PDL-1 positive tumors. Surprisingly, however, all 4 of the tumors that shrank in response to MEDI4736 treatment were HPV-negative. Thus, MEDI4736 is effective in treating HPV-negative tumors, despite the association of HPV-negative tumors with lower levels of the MEDI4736 antigen PDL-1.

Those skilled in the art will recognize, or be able to ascertain using no more than routine experimentation, many equivalents to the specific aspects of the disclosure described herein. Such equivalents are intended to be encompassed by the following claims.

Various publications are cited herein, the disclosures of which are incorporated by reference in their entireties.

Although the foregoing invention has been described in some detail by way of illustration and example for purposes of clarity of understanding, it will be obvious that certain changes and modifications can be practiced within the scope of the appended claims.

### Sequence Listing

SEQ ID NO: 1
>PCT/US2010/058007_77 Sequence 77 from
PCT/US2010/058007 Organism: Homo sapiens
EIVLTQSPGTLSLSPGERATLSCRASQRVSSSYLAWYQQKPGQAPRLLI
YDASSRATGIPDRFSGSGSGTDFTLTISRLEPEDFAVYYCQQYGSLPWT
FGQGTKVEIK
SEQ ID NO: 2
>PCT/US2010/058007_72 Sequence 72 from
PCT/US2010/058007 Organism: Homo sapiens
EVQLVESGGGLVQPGGSLRLSCAASGFTFSRYWMSWVRQAPGKGLEWVA
NIKQDGSEKYYVDSVKGRFTISRDNAKNSLYLQMNSLRAEDTAVYYCAR
EGGWFGELAFDYWGQGTLVTVSS
SEQ ID NO: 3-VH CDR1
>PCT/US2010/058007_73 Sequence 73 from
PCT/US2010/058007 Organism: Homo sapiens
RYWMS
SEQ ID NO: 4-VH CDR2
>PCT/US2010/058007_74 Sequence 74 from
PCT/US2010/058007 Organism: Homo sapiens
NIKQDGSEKYYVDSVKG
SEQ ID NO: 5-VH CDR3
>PCT/US2010/058007_75 Sequence 75 from
PCT/US2010/058007 Organism: Homo sapiens
EGGWFGELAFDY
SEQ ID NO: 6-VL CDR1
>PCT/US2010/058007_78 Sequence 78 from
PCT/US2010/058007 Organism: Homo sapiens
RASQRVSSSYLA
SEQ ID NO: 7-VL CDR2
>PCT/US2010/058007_79 Sequence 79 from
PCT/US2010/058007 Organism: Homo sapiens
DASSRAT
SEQ ID NO: 8-VL CDR3
>PCT/US2010/058007_80 Sequence 80 from
PCT/US2010/058007 Organism: Homo sapiens
QQYGSLPWT
SEQ ID NO: 9
>WO 2012/145493_1 Sequence 1 from WO 2012/145493
Organism: Mus musculus
DIVMTQSHKLMSTSVGDRVSITCKASQDVGTAVAWYQQKPGQSPKLLIY
WASTRHTGVPDRFTGSGSGTDFTLTISNVQSEDLADYFCQQDSSYPLTF
GAGTKVELK
SEQ ID NO: 10
>WO 2012/145493_2 Sequence 2 from WO 2012/145493
Organism: Mus musculus
EVKLQESGPSLVKPSQTLSLTCSVTGYSITSDYWNWIRKFPGNKLEYVG
YISYTGSTYYNPSLKSRISITRDTSKNQYYLQLNSVTSEDTATYYCARY
GGWLSPFDYWGQGTTLTVSS
SEQ ID NO: 11
>WO 2012/145493_3 Sequence 3 from WO 2012/145493
Organism: Mus musculus
DIVTTQSHKLMSTSVGDRVSITCKASQDVGTAVAWYQQKPGQSPKLLIY
WASTRHTGVPDRFTGSGSGTDFTLTISNVQSEDLADYFCQQDSSYPLTF
GAGTKVELK
SEQ ID NO: 12
>WO 2012/145493_4 Sequence 4 from WO 2012/145493
Organism: Mus musculus
EVQLQESGPGLVAPSQSLSITCTVSGFSLTTYSINWIRQPPGKGLEWLG
VMWAGGGTNSNSVLKSRLIISKDNSKSQVFLKMNSLQTDDTARYYCARY
YGNSPYYAIDYWGQGTSVTVSS
SEQ ID NO: 13
>WO 2012/145493_5 Sequence 5 from WO 2012/145493
Organism: Mus musculus
DIVMTQSPSSLAVSVGEKVSMGCKSSQSLLYSSNQKNSLAWYQQKPGQS
PKLLIDWASTRESGVPDRFTGSGSGTDFTLTISSVKAEDLAVYYCQQYY
GYPLTFGAGTKLELK
SEQ ID NO: 14
>WO 2012/145493_6 Sequence 6 from WO 2012/145493
Organism: Mus musculus
EVKLQESGPSLVKPSQTLSLTCSVTGYSIISDYWNWIRKFPGNKLEYLG
YISYTGSTYYNPSLKSRISITRDTSKNQYYLQLNSVTTEDTATYYCARR
GGWLLPFDYWGQGTTLTVS
SEQ ID NO: 15
>WO 2012/145493_7 Sequence 7 from WO 2012/145493
Organism: Mus musculus
DIVMTQSPAIMSASPGEKVTMTCSASSSIRYMHWYQQKPGTSPKRWISD
TSKLTSGVPARFSGSGSGTSYALTISSMEAEDAATYYCHQRSSYPWTFG
GGTKLEIK
SEQ ID NO: 16
>WO 2012/145493_8 Sequence 8 from WO 2012/145493
Organism: Mus musculus
EVKLQESGPSLVKPGASVKLSCKASGYTFTSYDINWVKQRPGQGLEWIG
WIFPRDNNTKYNENFKGKATLTVDTSSTTAYMELHSLTSEDSAVYFCTK
ENWVGDFDYWGQGTTLTLSS
SEQ ID NO: 17
>WO 2012/145493_81 Sequence 81 from WO 2012/
145493 Organism: Mus musculus
EIVLTQSPATLSLSPGERATLSCRASSSVSYTYWFQQKPGQAPRLLIYA
AFNRATGIPARFSGSGSGTDYTLTISSLEPEDFAVYYCQQWSNNPLTFG
QGTKVEIK
SEQ ID NO: 18
>WO 2012/145493_82 Sequence 82 from WO 2012/
145493 Organism: Mus musculus
EIVLTQSPATLSLSPGERATLSCRASSSVSYTYWFQQKPGQSPRPLIYA
AFNRATGIPARFSGSGSGTDYTLTISSLEPEDFAVYYCQQWSNNPLTFG
QGTKVEIK
SEQ ID NO: 19
>WO 2012/145493_83 Sequence 83 from WO 2012/
145493 Organism: Mus musculus
QIVLTQSPATLSLSPGERATLSCRASSSVSYTYWFQQKPGQSPRPLIYA
TFNLASGIPARFSGSGSGTSYTLTISRLEPEDFAVYYCQQWSNNPLTFG
QGTKVEIK
SEQ ID NO: 20
>WO 2012/145493_84 Sequence 84 from WO 2012/
145493 Organism: Mus musculus
DIQLTQSPSSLSASVGDRVTITCRASSGVSYIYWFQQKPGKAPKLLIYA
AFNLASGVPSRFSGSGSGTEYTLTISSLQPEDFATYYCQQWSNNPLTFG
QGTKVEIK
SEQ ID NO: 21
>WO 2012/145493_85 Sequence 85 from WO 2012/
145493 Organism: Mus musculus
DIQLTQSPSSLSASVGDRVTITCRASSGVSYIYWFQQKPGKAPKPLIYA
AFNLASGVPSRFSGSGSGTEYTLTISSLQPEDFATYYCQQWSNNPLTFG
QGTKVEIK
SEQ ID NO: 22
>WO 2012/145493_86 Sequence 86 from WO 2012/
145493 Organism: Mus musculus
DIQLTQSPS ILSASVGDRVTITCRASSSVSYIYWFQQKPGKAPKPLIY
ATFNLASGVPSRFSGSGSGTSYTLTISSLQPEDFATYYCQQWSNNPLTF
GQGTKVEIK
SEQ ID NO: 23
>WO 2012/145493_90 Sequence 90 from WO 2012/
145493 Organism: Mus musculus
QVQLVQSGAEVKKPGASVKVSCKASGYTFPDYYMNWVRQAPGQGLEWMGD
IDPNYGGTNYAQKFQGRVTMTRDTSISTAYMELSRLRSDDTAVYYCARGA
LTDWGQGTMVTVSS
SEQ ID NO: 24
>WO 2012/145493_91 Sequence 91 from WO 2012/
145493 Organism: Mus musculus
QVQLVQSGAEVKKPGASVKVSCKASGYTFPDYYMNWVRQAPGQSLEWMG
DIDPNYGGTNYNQKFQGRVTMTRDTSISTAYMELSRLRSDDTAVYYCAR
GALTDWGQGTMVTVSS
SEQ ID NO: 25
>WO 2012/145493_92 Sequence 92 from WO 2012/
145493 Organism: Mus musculus
EVQLVQSGAEVKKPGASVKVSCKASGYTFPDYYMNWVRQAPGQSLEWMG
DIDPNYGGTNYNQKFQGRVTMTVDRSSSTAYMELSRLRSDDTAVYYCAR
GALTDWGQGTMVTVSS
SEQ ID NO: 26
>WO 2012/145493_93 Sequence 93 from WO 2012/
145493 Organism: Mus musculus
EVQLVESGGGLVQPGRSLRLSCTASGYTFPDYYMNWVRQAPGKGLEWVG
DIDPNYGGTTYAASVKGRFTISVDRSKSIAYLQMSSLKTEDTAVYYCTR
GALTDWGQGTMVTVSS
SEQ ID NO: 27
>WO 2012/145493_94 Sequence 94 from WO 2012/
145493 Organism: Mus musculus
EVQLVE SGGGLVQPGRSLRLSCTASGYTFPDYYMNWVRQAPGKGLEWV
GDIDPNYGGTTYNASVKGRFTISVDRSKSIAYLQMSSLKTEDTAVYYCA
RGALTDWGQGTMVTVSS
SEQ ID NO: 28
>WO 2012/145493_95 Sequence 95 from WO 2012/
145493 Organism: Mus musculus
EVQLVE SGGGLVQPGRSLRLSCTASGYTFPDYYMNWVRQAPGKGLEWV
GDIDPNYGGTTYNQSVKGRFTISVDRSKSIAYLQMSSLKTEDTAVYYCA
RGALTDWGQGTMVTVSS
SEQ ID NO: 29
>WO 2012/145493_11 Sequence 11 from WO 2012/
145493 Organism: Mus musculus
DIQMTQFPSSLCASQGGKVTVTCKASQDINNYMAWYQHKPGKGPRLLIH
YTSTLLSGIPSRFSGSGSGRDYSFSISNLEPEDIATYYCLQYDNLWTFG
GGTKLEIK
SEQ ID NO: 30
>WO 2012/145493_12 Sequence 12 from WO 2012/
145493 Organism: Mus musculus
EVQLQQSGPVLVKPGASVKMSCKASGYTFTDYYMNWVKQSHGKSLEWIG
NINPYNGGTTYNQKFKGKATLTVDKSSRTAYMEINSLTSEDSAVYYCAR
GRIYDGSLDYWGQGTALTVSS
SEQ ID NO: 31
>WO 2012/145493_13 Sequence 13 from WO 2012/
145493 Organism: Mus musculus
DIVMTQSQKFMSTSVGDRVSVTCKASQSVDTNVAWYQQKPGQSPKALIF
SASYRYSGVPDRFTGSGSGTDFTLTINSVQSEDLAEYFCQQYNSYPYTF
GSGTKLEIK
SEQ ID NO: 32
>WO 2012/145493_14 Sequence 14 from WO 2012/
145493 Organism: Mus musculus
QVQLQQSGAELAKPGASVRLSCKASGYTFTNYWMHWVKQRPGQGLEWIG
IHNPSSGFTTYNQNFKDKATLTADKSSNTAYMQLSSLTYEDSAVYFCAR
EDYDVDYWGQGTTLTVSS
SEQ ID NO: 33
>WO 2012/145493_15 Sequence 15 from WO 2012/145493
Organism: Mus musculus
QIVLTQSPALMSASPGEKVTMTCSASSSVSYMYWYQQKPRSSPKPWIYL
TSNLASGVPARFSGSGSGTSYSLTISSMEAEDAATYYCQQWSSNPFTFG
SGTKLEIK
SEQ ID NO: 34
>WO 2012/145493_16 Sequence 16 from WO 2012/
145493 Organism: Mus musculus
EVQLVESGGGLVKPGGSLKLSCAASGFTFSDYGMHWVRQAPEKGLEWVA
YISSGSYTIYYTDTVKGRFTISRDNAKNTLFLQMTSLRSEDTAMYYCAR
RGYGSFYEYYFDYWGQGTTLTVSS
SEQ ID NO: 35
>WO 2012/145493_97 Sequence 97 from WO 2012/
145493 Organism: Mus musculus
EIVLTQSPATLSLSPGERATLSCRASSSVSYMYWYQQKPGQAPRLLIYL
ASNRATGIPARFSGSGSGTDYTLTISSLEPEDFAVYYCQQWSSNPFTFG
QGTKLEIK
SEQ ID NO: 36
>WO 2012/145493_98 Sequence 98 from WO 2012/
145493 Organism: Mus musculus
QIVLTQSPATLSLSPGERATLSCSASSSVSYMYWYQQKPGQAPRLLTYL
TSNRATGIPARFSGSGSGTDYTLTISSLEPEDFAVYYCQQWSSNPFTFG
QGTKLEIK
SEQ ID NO: 37
>WO 2012/145493_99 Sequence 99 from WO 2012/
145493 Organism: Mus musculus
DIQLTQSPSSLSASVGDRVTITCRASSSVSYMYWYQQKPGKAPKLLIYL
ASNLASGVPSRFSGSGSGTEYTLTISSLEPEDFATYYCQQWSSNPFTFG
QGTKLEIK
SEQ ID NO: 38
>WO 2012/145493_100 Sequence 100 from WO 2012/
145493 Organism: Mus musculus
QTQLTQSPSSLSASVGDRVTITCSASSSVSYMYWYQQKPGKAPKLLTYL
TSNLASGVPSRFSGSGSGTEYTLTISSLEPEDFATYYCQQWSSNPFTFG
QGTKLEIK
SEQ ID NO: 39
>WO 2012/145493_104 Sequence 104 from WO 2012/
145493 Organism: Mus musculus
EVQLVESGGGLVQPGGSLRLSCAASGFTFSDYGMHWVRQAPGKGLEWVS
YISSGSSTIYYADSVKGRFTISRDNAKNTLYLQMSSLRAEDTAVYYCAR
RGYGSFYEYYFDYWGQGTTVTVSS
SEQ ID NO: 40
>WO 2012/145493_105 Sequence 105 from WO 2012/
145493 Organism: Mus musculus
EVQLVESGGGLVQPGGSLRLSCAASGFTFSDYGMHWVRQAPGKGLEWVA
YISSGSYTIYYADSVKGRFTISRDNAKNTLYLQMSSLRAEDTAVYYCAR
RGYGSFYEYYFDYWGQGTTVTVSS
SEQ ID NO: 41
>WO 2012/145493_106 Sequence 106 from WO 2012/
145493 Organism: Mus musculus
EVQLVESGGGLVQPGGSLRLSCAASGFTFSDYGMHWVRQAPGKGLEWVA
YISSGSYTIYSADSVKGRFTISRDNAKNTLYLQMSSLRAEDTAVYYCAR
RGYGSFYEYYFDYWGQGTTVTVSS
SEQ ID NO: 42
>WO 2012/145493_107 Sequence 107 from WO 2012/
145493 Organism: Mus musculus
QVQLVQSGAEVKKPGASVKVSCKASGFTFSDYGMHWVRQAPGQRLEWMG
YISSGSSTIYYSQKFQGRVTITRDNSASTLYMELSSLRSEDTAVYYCAR
RGYGSFYEYYFDYWGQGTTLTVSS
SEQ ID NO: 43
>WO 2012/145493_108 Sequence 108 from WO 2012/
145493 Organism: Mus musculus
EVQLVQSGAEVKKPGASVKVSCAASGFTFSDYGMHWVRQAPGQRLEWMG
YISSGSYTIYYSQKFQGRVTITRDNSASTLYMELSSLRSEDTAVYYCAR
RGYGSFYEYYFDYWGQGTTLTVSS
SEQ ID NO: 44
>WO 2012/145493_109 Sequence 109 from WO 2012/
145493 Organism: Mus musculus
EVQLVQSGAEVKKPGASVKVSCAASGFTFSDYGMHWVRQAPGQRLEWVA
YISSGSYTIYYSQKFQGRVTITRDNSASTLYMELSSLRSEDTAVYYCAR
RGYGSFYEYYFDYWGQGTTLTVSS
SEQ ID NO: 45
>WO 2012/145493_9 Sequence 9 from WO 2012/145493
Organism: Mus musculus
QIVLSQSPAILSASPGEKVTMTCRASSSVSYIYWFQQKPGSSPKPWIYA
TFNLASGVPARFSGSGSGTSYSLTISRVETEDAATYYCQQWSNNPLTFG
AGTKLELK
SEQ ID NO: 46
>WO 2012/145493_10 Sequence 10 from WO 2012/
145493 Organism: Mus musculus
EVQLQQSGPDLVTPGASVRISCQASGYTFPDYYMNWVKQSHGKSLEWIG
DIDPNYGGTTYNQKFKGKAILTVDRSSSTAYMELRSLTSEDSAVYYCAR
GALTDWGQGTSLTVSS

1. A method of treating a cancer comprising administering a PDL-1 antagonist to a human patient having cancer, wherein the cancer is HPV-negative.

2. The method of claim 1, wherein the PDL-1 antagonist is an anti-PDL-1 antibody or antigen-binding fragment thereof.

3. The method of claim 1, wherein the PDL-1 antagonist inhibits the interaction of PDL-1 and PD-1.

4-7. (canceled)

8. The method of claim 1, wherein the antagonist is MEDI4736 or an antigen-binding fragment thereof.

9. The method of claim 8, further comprising determining if the cancer is HPV-negative.

10. The method of claim 8, wherein the administration reduces tumor growth.

11. The method of claim 8, wherein the administration decreases tumor size.

12-17. (canceled)

18. The method of claim 8, wherein about 0.1, about 0.3, about 1, about 3, about 10, or about 15 mg/kg MEDI4736 or an antigen-binding fragment thereof is administered.

19. The method of claim 18, wherein about 10 mg/kg MEDI4736 or an antigen-binding fragment thereof is administered.

20-22. (canceled)

23. The method of claim 8, wherein the administration results in a partial response.

24. The method of claim 8, wherein the administration results in a complete response.

25. The method claim 8, wherein the cancer squamous cell carcinoma of the head and neck (SCCHN).

26. The method of claim 25, wherein the cancer is oropharyngeal squamous cell carcinoma.

27. The method of claim 8, wherein the tumor is refractory to at least one chemotherapeutic agent.
